(19) United States
(12) Patent Application Publication
(10) Pub. No.: US 2003/0113301 A1
(43) Pub. Date: Jun. 19, 2003

(54) Muscle cells and their use in cardiac repair

(76) Inventors: Albert Edge, Cambridge, MA (US); Jonathan Dinsmore, Brookline, MA (US)

Correspondence Address: Monica R. Gerber, M.D., Ph.D., Choate, Hall & Stewart, Exchange Place, 53 State Street, Boston, MA, 02109, US

(21) Appl. No.: 10/105,035

(22) Filed: Mar. 21, 2002

Related U.S. Application Data
(63) Continuation-in-part of application No. 09/624,885, filed on Jul. 24, 2000.
(60) Provisional application No. 60/145,849, filed on Jul. 23, 1999.

(51) Int. Cl. A61K 48/00

(52) U.S. Cl. 424/93.21; 424/93.7

(57) ABSTRACT

Muscle cells and methods for using the muscle cells are provided. In one embodiment, the invention provides transplantable skeletal muscle cell compositions and their methods of use. In one embodiment, the muscle cells can be transplanted into patients having disorders characterized by insufficient cardiac function, e.g., congestive heart failure, in a subject by administering the skeletal myoblasts to the subject. The muscle cells can be autologous, allogeneic, or xenogeneic to the recipient.

### RELATED APPLICATIONS

This application claims priority to U.S. Provisional Application No. 60/145,849, filed on Jul. 23, 1999, and to U.S. National application Ser. No. 09/624,885, filed on Jul. 24, 2000, both of which are incorporated herein in their entirety.

### BACKGROUND OF THE INVENTION

Heart disease is the predominant cause of disability and death in all industrialized nations. Cardiac disease can lead to decreased quality of life and long term hospitalization. In addition, in the United States, it accounts for about 335 deaths per 100,000 individuals (approximately 40% of the total mortality) overshadowing cancer, which follows with 183 deaths per 100,000 individuals. Four categories of heart disease account for about 85-90% of all cardiac-related deaths. These categories are: ischemic heart disease, hypertensive heart disease and pulmonary hypertensive heart disease, valvular disease, and congenital heart disease. Ischemic heart disease, in its various forms, accounts for about 60-75% of all deaths caused by heart disease. In addition, the incidence of heart failure is increasing in the United States. One of the factors that renders ischemic heart disease so devastating is the inability of the cardiac muscle cells to divide and repopulate areas of ischemic heart damage. As a result, cardiac cell loss as a result of injury or disease is irreversible.

Human to human heart transplants have become the most effective form of therapy for severe heart damage. Many transplant centers now have one-year survival rates exceeding 80-90% and five-year survival rates above 70% after cardiac transplantation. Heart transplantation, however, is severely limited by the scarcity of suitable donor organs. In addition to the difficulty in obtaining donor organs, the expense of heart transplantation prohibits its widespread application. Another unsolved problem is graft rejection. Foreign hearts are poorly tolerated by the recipient and are rapidly destroyed by the immune system in the absence of immunosuppressive drugs. While immunosuppressive drugs may be used to prevent rejection, they also block desirable immune responses such as those against bacterial and viral infections, thereby placing the recipient at risk of infection. Infections, hypertension, and renal dysfunction caused by cyclosporin, rapidly progressive coronary atherosclerosis, and immunosuppressant-related cancers have been major complications however.

Cellular transplantation has been the focus of recent research into new means of repairing cardiac tissue after myocardial infarctions. A major problem with transplantation of adult cardiac myocytes is that they do not proliferate in culture. (Yoon et al. (1 995) Tex. Heart Inst. J. 22:119). To overcome this problem, attention has focused on the possible use of skeletal myoblasts. Skeletal muscle tissue contains satellite cells which are capable of proliferation. However, methods of purifying and growing these cells are complicated. There is a clear need, therefore, to address the limitations of the current heart transplantation therapies in the treatment of heart disease.

### SUMMARY OF THE INVENTION

To overcome the limitations of the current heart repair methodologies, the present invention provides isolated muscle cells. In a preferred embodiment, the invention pertains to skeletal myoblasts, compositions including the skeletal myoblasts, and methods for transplanting skeletal myoblasts into subjects. In addition, the invention pertains to cardiomyocytes, methods for inducing the proliferation of cardiomyocytes, and methods for transplanting cardiomyocytes to subjects. The present invention offers numerous advantages over the cells and methods of the prior art.

In one aspect, the invention provides a method for preparing a transplantable muscle cell composition comprising skeletal myoblast cells and fibroblast cells comprising culturing the composition on a surface coated with poly-L-lysine and laminin in a medium comprising EGF such that the transplantable composition is prepared. Preferably, the cells are permitted to double less than about 10 times in vitro prior to transplantation such that the fibroblast to myoblast ratio is approximately 1:2 to 1:1.

In one aspect, the invention provides a transplantable composition comprising skeletal myoblast cells and fibroblast cells and, in one embodiment, can comprise from about 20% to about 70% myoblasts and, preferably, about 40-60% myoblasts or about 50% myoblasts. In another embodiment, the transplantable composition comprises at least about 20%, 25%, 30%, 35%, 40%, 45%, 50%, 55%, 60%, 65%, 70%, 75%, 80%, 85%, 90%, 95%, 96%, 97%, 98% or 99% myoblasts.

The muscle cells of the invention may be cultured in vitro prior to transplantation and are preferably cultured on a surface coated with poly-L-lysine and laminin in a medium comprising EGF. Alternatively, the surface can be coated with collagen and the composition cultured in a medium comprising FGF.

The muscle cells of the invention preferably engraft into cardiac tissue after transplantation into a subject. The muscle cells of the invention can endogenously express an angiogenic factor, or can be administered in the form of a composition which comprises an angiogenic factor, or the muscle cells of the invention can be engineered to express an angiogenic gene product in order to induce angiogenesis in the recipient heart.

The invention also provides for modifying, masking, or eliminating an antigen on the surface of a cell in the composition such that upon transplantation of the composition into a subject lysis of the cell is inhibited. In one embodiment, PT85 or W6/32 is used to mask an antigen.

The invention further provides a method for treating a condition in a subject characterized by damage to cardiac tissue comprising transplanting a composition comprising skeletal myoblast cells and fibroblast cells into a subject such that the condition is thereby treated.

The invention further provides a method for treating myocardial ischemic damage comprising transplanting a composition comprising skeletal myoblast cells and, optionally, fibroblast cells into a subject such that the myocardial ischemic damage is thereby treated. According to certain embodiments of the invention at least a portion of the skeletal myoblasts, or cells to which the skeletal myoblasts give rise, survive in the heart after delivery and express therein a marker characteristic of skeletal myoblast survival or differentiation.

In one embodiment, skeletal myoblast cells of the invention can be induced to become more like cardiac cells. In a preferred embodiment, a cardiac cell phenotype in a skeletal myoblast is promoted by recombinantly expressing a cardiac cell gene product in the myoblast so that the cardiac cell phenotype is promoted. In one embodiment, the gene product is a GATA transcription factor and, preferably is GATA4 or GATA6.

### BRIEF DESCRIPTION OF THE DRAWING

FIG. 1 shows that muscle cells that undergo fewer population doublings result in better survival after transplantation. FIG. 1A is a photograph of transplanted cells which were sorted prior to transplantation, while FIG. 1B is a photograph of transplanted cells which were not sorted and were only allowed to undergo several population doublings in vitro prior to transplantation.

FIG. 2 shows that vessel formation (angiogenesis) occurs after transplantation of muscle cells. FIGS. 2A (lower power) and 2B (higher power) shows staining of such a graft for factor VIII at three weeks post transplantation. Vessels can be seen in the center of the graft.

FIGS. 3-4 show that transplanted animals (myoblast and fibroblast) showed improvements in diastolic pressure-volume as compared to nontransplanted control animals.

FIGS. 5A-5F show myoblast survival in infarcted myocardium at 9 days post-implantation. FIG. 5 is the infarcted left ventricular free wall of a rat under increasing magnification, with trichome staining (A, B, and C) and immunohistochemical staining for myogenin, a nuclear transcription factor unique to skeletal myoblasts (D, E, and F). The encircled area identifies the region of cell implantation. Arrows highlight two grafts within the infarct region.

FIG. 6 shows that transplanted post-myocardial infarction animals (myoblast and fibroblast) showed improvements in systolic pressure-volume as compared to nontransplanted control animals. FIG. 6 is the maximum exercise capacity determined prior to cell therapy (1 week post-MI), 3 weeks post-implantation, and 6 weeks post-implantation. Non-infarcted control animals, dashed bar; MI animals, dark bar; MI+ animals, light bar; *, p<0.05 vs. 0 weeks (pre-therapy); #, p<0.05 vs. MI.

FIGS. 7A-7B show that transplanted post-myocardial infarction animals (myoblast and fibroblast) showed improvements in diastolic pressure-volume as compared to nontransplanted control animals. FIG. 7 is the systolic pressure-volume relationships at three weeks post-cell therapy (A) and six weeks post-cell therapy (B). Control hearts, dashed line; MI hearts, dark boxes, MI+ hearts, light boxes, *, p<0.05 vs. control.

FIGS. 8A-8B show that transplanted post-myocardial infarction animals (myoblast and fibroblast) show no significant decrease in infarct wall thickness as compared to nontransplanted control animals. FIG. 8 is the diastolic pressure-volume relationships at three weeks post-cell therapy (A) and six weeks post-cell therapy (B). Control hearts, dashed line; MI hearts, dark boxes, MI+ hearts, light boxes, *, p<0.05 vs. control, #, p<0.05 vs. MI.

FIG. 9 shows a histogram plot of FACS analysis performed prior to transplantation. Myoblasts were stained with N-CAM antibody and then subjected to FACS analysis. The histogram plot shows the intensity and homogeneity of staining with N-CAM versus and isotype matched negative control sample.

FIG. 10 is a micrograph showing trichrome and MY-32 staining of the graft. Panel (A) shows an area of the graft in a section stained with trichrome. Panel (B) shows an adjacent section that was stained with MY-32. The transplant derived myofibers can be identified by the red staining in trichrome and the dark blue staining in the MY-32 stain. Asterisks (*) mark areas of host myocardial fibers. Scale bar=50 microns.

FIG. 11 is a micrograph showing CD-31 staining of the graft. An antibody to human CD-31 was used to stain graft sections. Panel (A) shows a representative micrograph in the area of the graft. The dotted line demarcates the border area between the transplant and the adjacent scar. Panel (B) shows the results from quantitative counts to compare the number of CD-31 vessels at the graft and in the adjacent scar. Scale bar=100 microns.

FIG. 12 is a micrograph showing a trichrome stain of surviving skeletal myofibers in patient heart. This area extends up from the epicardial surface of the myocardium into the epicardial fat. Blue stain represents collagen fibrils and red patches represent surviving myofibers. The boxed area is shown in FIG. 13 at higher magnification. Total magnification for this image=50×.

FIG. 13 is a micrograph showing a trichrome stain of surviving skeletal myofibers shown at 200×magnification. The blue staining area represents an area of collagen fibril deposition typical of scarred myocardium. The red stained areas marked by arrows show the myofibers, some of which show a striated appearance.

FIG. 14 is a micrograph showing staining of skeletal muscle fibers with skeletal muscle specific myosin. Same area as shown in FIG. 12. 100×magnification.

FIG. 15 is a micrograph showing muscle specific myosin staining of surviving skeletal muscle fibers in transplanted heart. The myofibers are shown in the myocardium close to the epicardial surface. 50×magnification.

### DETAILED DESCRIPTION OF CERTAIN PREFERRED EMBODIMENTS OF THE INVENTION

The invention features isolated muscle cells, e.g., skeletal myoblasts, cardiomyocytes, or compositions comprising skeletal myoblasts or cardiomyocytes and their methods of use. In certain embodiments, the invention provides isolated skeletal myoblasts and populations of isolated skeletal myoblasts suitable for introduction into a recipient. The populations and compositions may further include fibroblasts. In most instances the fibroblasts are isolated from muscle samples although they may be isolated from other sources such as skin tissue or may be cell lines. For purposes of description herein, when the term “muscle cell composition” refers to a composition comprising fibroblasts, unless otherwise specified or clear from context, the term will be taken to include fibroblasts isolated from any source, not limited to muscle. The invention further provides methods of transplanting such cells. In addition, the invention provides methods of isolating and expanding skeletal myoblasts, fibroblasts, adult cardiomyocytes, isolated cardiomyocytes, populations of isolated, expanded cardiomyocytes, compositions including cardiomyocytes, and methods for transplanting cardiomyocytes into a recipient. These cells can be transplanted, for example, into recipient subjects that have dysfunctional and/or damaged heart tissue. Such dysfunction or damage may result from a wide variety of disorders such as ischemic heart disease, hypertensive heart disease and pulmonary hypertensive heart disease (cor pulmonale), valvular disease, congenital heart disease, dilated cardiomyopathy, hypertrophic cardiomyopathy, myocardidtis, viral infection, immune-mediated conditions, wounds, exogenous compounds such as drugs or toxins (by exogenous compound is meant a compound that is not found naturally within a subject's body), or any condition which leads to heart failure, e.g., which is characterized by insufficient cardiac function.

I. Definitions

For the sake of convenience, certain terms used throughout the specification are collected here.

As used herein, the term “isolated” refers to a cell which has been separated from its natural environment. This term includes gross physical separation of the cell from its natural environment, e.g., removal from the donor. Preferably “isolated” includes alteration of the cell's relationship with the neighboring cells with which it is in direct contact by, for example, dissociation. The term “isolated” does not refer to a cell which is in a tissue section, is cultured as part of a tissue section, or is transplanted in the form of a tissue section. When used to refer to a population muscle cells, the term “isolated” includes populations of cells which result from proliferation of the isolated cells of the invention.

The terms “skeletal myoblasts” and “skeletal myoblast cells” are used interchangeably herein and refer to a precursor of myotubes and skeletal muscle fibers. The term “skeletal myoblasts” also includes satellite cells, mononucleate cells found in close contact with muscle fibers in skeletal muscle. Satellite cells lie near the basal lamina of skeletal muscle myofibers and can differentiate into myofibers. As discussed herein, preferred compositions comprising skeletal myoblasts lack detectable myotubes and muscle fibers. The term “cardiomyocyte” includes a muscle cell which is derived from cardiac muscle. Such cells have one nucleus and are, when present in the heart, joined by intercalated disc structures.

As used herein, the term “engrafts” includes the incorporation of transplanted muscle cells or muscle cell compositions of the invention into heart tissue with or without the direct attachment of the transplanted cell to a cell in the recipient heart, (e.g., by the formation desmosomes or gap junctions) such that the cells enhance cardiac function, e.g., by increasing cardiac output.

As used herein the term “angiogenesis” includes the formation of new capillary vessels in the heart tissue into which the muscle cells of the invention are transplanted. Preferably, the muscle cells of the invention, when transplanted into an ischemic zone, enhance angiogeniesis. This angiogenesis can occur, e.g., as a result of the act of transplanting the cells, as a result of the secretion of angiogenic factors from the muscle cells, and/or as a result of the secretion of endogenous angiogenic factors from the heart tissue.

As used herein, the terms “approximately” or “about” in reference to a number are taken to include numbers that fall within a range of 2.5% in either direction (greater than or less than) the number.

As used herein, the term “essentially free of” indicates that the relevant item (e.g., cell) is undetectable using either a detection procedure described herein or a comparable procedure known to one of ordinary skill in the art.

As used herein the phrase “more like cardiac cells” includes skeletal muscle cells which are made to more closely resemble cardiac muscle cells in phenotype. Such cardiac-like cells can be characterized, e.g., by a change in their physiology (e.g., they may have a slower twitch phenotype, a slower shortening velocity, use of oxidative phosphorylation for ATP production, expression of cardiac forms of contractile proteins, higher mitochondrial content, higher myoglobin content, and greater resistance to fatigue than skeletal muscle cells) and/or the production of molecules which are normally not produced by skeletal muscle cells or which are normally produced in low amounts by skeletal muscle cells (e.g., those proteins produced from genes encoding the myocardial contractile apparatus and the Ca++ATPase associated with cardiac slow twitch, phospholamban, and/or β myosin heavy molecules).

As used herein the phrase “GATA transcription factor” includes members of the GATA family of zinc finger transcription factors. GATA transcription factors play important roles in the development of several mesodermally derived cell lineages. Preferably, GATA transcription factors include GATA-4 and/or GATA-6. The GATA-6 and GATA-4 proteins share high-level amino acid sequence identity over a proline-rich region at the amino terminus of the protein that is not conserved in other GATA family members.

As used herein, the term “antibody” is intended to include immunoglobulin molecules and immunologically active portions of immunoglobulin molecules, i.e., molecules that contain an antigen binding site which specifically binds (immunoreacts with) an antigen, such as Fab and F(ab′)₂ fragments. The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody compositions thus typically display a single binding affinity for a particular antigen with which it immunoreacts.

As used herein, a cell is “derived from” a subject or sample if the cell is obtained from the sample or subject or if the cell is the progeny or descendant of a cell that was obtained from the sample or subject. A cell that is derived from a cell line is a member of that cell line or is the progeny or descendant of a cell that is a member of that cell line. A cell derived from an organ, tissue, individual, cell line, etc., may be modified in vitro after it is obtained. Such a cell is still considered to be derived from the original source.

As used herein, the terms “myoblast survival” or “fibroblast survival” within the heart is intended to indicate any of the following and combinations thereof: (1) survival of the myoblasts or fibroblasts themselves; (2) survival of cells into which the myoblasts or fibroblasts differentiate; (3) survival of progeny of the myoblasts or fibroblasts.

As used herein, the phrase “cardiac damage” or “disorder characterized by insufficient cardiac function” includes any impairment or absence of a normal cardiac function or presence of an abnormal cardiac function. Abnormal cardiac function can be the result of disease, injury, and/or aging. As used herein, abnormal cardiac function includes morphological and/or functional abnormality of a cardiomyocyte or a population of cardiomyocytes. Non-limiting examples of morphological and functional abnormalities include physical deterioration and/or death of cardiomyocytes, abnormal growth patterns of cardiomyocytes, abnormalities in the physical connection between cardiomyocytes, under- or over-production of a substance or substances by cardiomyocytes, failure of cardiomyocytes to produce a substance or substances which they normally produce, and transmission of electrical impulses in abnormal patterns or at abnormal times. Abnormal cardiac function is seen with many disorders including, for example, ischemic heart disease, e.g., angina pectoris, myocardial infarction, chronic ischemic heart disease, hypertensive heart disease, pulmonary heart disease (cor pulmonale), valvular heart disease, e.g., rheumatic fever, mitral valve prolapse, calcification of mitral annulus, carcinoid heart disease, infective endocarditis, congenital heart disease, myocardial disease, e.g., myocarditis, dilated cardiomyopathy, hypertensive cardiomyopathy, cardiac disorders which result in congestive heart failure, and tumors of the heart, e.g., primary sarcomas and secondary tumors.

As used herein, the phrase “myocardial ischemia” includes a lack of oxygen flow to the heart which results in “myocardial ischemic damage.” As used herein, the phrase “myocardial ischemic damage” includes damage caused by reduced blood flow to the myocardium. Non-limiting examples of causes of myocardial ischemia and myocardial ischemic damage include: decreased aortic diastolic pressure, increased intraventricular pressure and myocardial contraction, coronary artery stenosis (e.g., coronary ligation, fixed coronary stenosis, acute plaque change (e.g., rupture, hemorrhage), coronary artery thrombosis, vasoconstriction), aortic valve stenosis and regurgitation, and increased right atrial pressure. Non-limiting examples of adverse effects of myocardial ischemia and myocardial ischemic damage include: myocyte damage (e.g., myocyte cell loss, myocyte hypertrophy, myocyte cellular hyperplasia), angina (e.g., stable angina, variant angina, unstable angina, sudden cardiac death), myocardial infarction, and congestive heart failure. Damage due to myocardial ischemia may be acute or chronic, and consequences may include scar formation, cardiac remodeling, cardiac hypertrophy, wall thinning, and associated functional changes. The existence and etiology of acute or chronic myocardial damage and/or myocardial ischemia may be diagnosed using any of a variety of methods and techniques well known in the art including, e.g., non-invasive imaging, angiography, stress testing, assays for cardiac-specific proteins such as cardiac troponin, and clinical symptoms. These methods and techniques as well as other appropriate techniques may be used to determine which subjects are suitable candidates for the treatment methods described herein.

The term “treating” as used herein includes reducing or alleviating at least one adverse effect or symptom of myocardial damage or dysfunction. On particular, the term applies to treatment of a disorder characterized by myocardial ischemia, myocardial ischemic damage, cardiac damage or insufficient cardiac function. Adverse effects or symptoms of cardiac disorders are numerous and well-characterized. Non-limiting examples of adverse effects or symptoms of cardiac disorders include: dyspnea, chest pain, palpitations, dizziness, syncope, edema, cyanosis, pallor, fatigue, and death. For additional examples of adverse effects or symptoms of a wide variety of cardiac disorders, see Robbins, S. L. et al. (1984) Pathological Basis of Disease (W. B. Saunders Company, Philadelphia) 547-609; Schroeder, S. A. et al. eds. (1992) Current Medical Diagnosis & Treatment (Appleton & Lange, Connecticut) 257-356.

II. Muscle Cells of the Invention

Cells that can be transplanted using the instant methods include skeletal myoblasts and cardiomyocytes. The cells used in this invention can be derived from a suitable mammalian source, e.g., from pigs or from humans. They can be, for example, autologous, allogeneic, or xenogeneic to the subject into which they are transplanted. In preferred embodiments, the cells are human cells and are used for transplantation into the same individual from which they were derived or are used for transplantation into an allogeneic subject. Cells for use in the invention can be derived from a donor of any gestational age, e.g., they can be adult cells, neonatal cells, fetal cells, embryonic stem cells, or muscle cells derived from embryonic stem cells (e.g., as described by Klug et al. 1996. J. Clin. Invest.98:216).

Standard methods can be used to prepare the muscle cells of the invention. Muscle cells can be isolated from donor muscle tissue using standard methods, e.g., mechanical and/orenzymatic digestion. For example, in preparing skeletal myoblasts, skeletal muscle cells can be isolated from, for example, limb muscle such as the quadriceps, or from another appropriate muscle (e.g., a hind leg muscle of an animal); cardiomyocytes can be prepared from heart tissue.

If desired, the site from which the muscle tissue is obtained may be stimulated prior to tissue harvest in order to increase the number of myoblasts. Such stimulation may be mechanical and/or by treatment with compounds such as growth factors. According to certain embodiments of the invention between approximately 0.5 and 2.0 grams of tissue are isolated. According to certain embodiments of the invention between approximately 2 and 4 grams of tissue are isolated. According to certain embodiments of the invention between approximately 4 and 6 grams of tissue are isolated. Tissue can be cut into pieces, e.g., with surgical blade before or after placing the tissue in digestion medium. If desired, rather than digesting the tissue at this stage it may be cryopreserved for future use. The biopsy pieces can be teased into fine fragments, e.g., using the needle tips of two tuberculin syringe needle assemblies. Connective tissue may be removed, e.g., using visual inspection. If desired, such tissue may be cultured separately in order to obtain fibroblasts.

According to certain embodiments of the invention the digestion medium comprises protease. In some embodiments, only a single protease is used; in other embodiments, at least two proteases are used, either in a sequence of separate digestion steps (e.g., alternating), or in combination. Appropriate proteases may include any of the following: carboxypeptidase, caspase, chymotrypsin, collagenase, elastase, endoproteinase, leucine aminopeptidase, papain, pronase, and trypsin (available, e.g., from Sigma Chemical Corporation (St. Louis, Mo.). According to certain embodiments of the invention EDTA is present in the digestion medium. A range of different protease concentrations and digestion temperatures may be used such as are well known to one of ordinary skill in the art. A range of digestion periods may be used. In general, 37 degrees C. is an appropriate temperature, and between 5 and 15 minutes or between 8 and 10 minutes is an appropriate digestion period. Procedures such as vortexing may be used to aid in separating cells from tissue.

In those embodiments of the invention in which a sequence of digestion steps is used, any of a variety of procedures may be followed. For example, tissue may be maintained in digestion medium for a period of time following which the digestion medium may be removed (e.g., after spinning down the cells and tissue) and replaced with fresh medium. Alternately, cells that have been released from the tissue mass may be collected at each digestion step. This step can be repeated as appropriate to maximize myoblast purification. The absolute and relative yield of myoblasts, fibroblasts, etc., at each step may be estimated, e.g., by visual inspection. Isolated cells can be pooled into groups and expanded as described below. According to certain embodiments of the invention in which muscle cells are collected in separate pools at each of a number of digestion steps, it may be desirable to select certain pools for combination and expansion depending, for example, upon the percentage of myoblasts and fibroblasts in each pool. For example, it may be desirable to perform a sequence of approximately 10 to 12 digestions steps. It may be desirable to pool, e.g., the cells isolated during steps 2 through 7, steps 3 through 8, steps 4 through 9, etc. In general, selection of the appropriate populations to pool will depend on the absolute and relative cell numbers in each pool, the total number of cells desired, and the cell types ultimately desired for the transplantable composition. According to certain embodiments of the invention the cells may be sorted, e.g., using fluorescence activated cell sorting (FACS) as is well known in the art. Sorting may be performed following the initial harvest, e.g., before expanding the cells in culture, or at any stage during the expansion process. Sorting may be used to select populations of cells having desired percentages of myoblasts or fibroblasts. Sorting may be used to reduce the number of endothelial cells.

The invention further provides transplantable muscle cell compositions. Preferably, such compositions comprise muscle cells that have been cultured in vitro for less than about 50 population doublings prior to transplantation. In one embodiment, the muscle cells are permitted to undergo less than about 20 population doublings in vitro prior to transplantation. In one embodiment, the muscle cells are permitted to undergo less than about 10 population doublings in vitro prior to transplantation. In another embodiment, the muscle cells are permitted to undergo less than about 5 population doublings in vitro prior to transplantation. In yet another embodiment, the muscle cells of the invention are permitted to undergo between about I and about 5 population doublings in vitro prior to transplantation. In another embodiment, the muscle cells of the invention are permitted to undergo between about 2 and about 4 population doublings in vitro prior to transplantation. The optimal number of doublings may vary depending upon the mammal from which the cells were isolated; the optimal numbers of doublings set forth here are for human cells. A rough calculation for cells from other species can be made by comparing the number of doublings before senescence is reached for that species with the number of doublings before senescence is reached in human cells and adjusting the number of doublings accordingly. For example, if cells from a different species go through about half as many doublings as human cells before reaching senescence, then the preferred number of population doublings for that species would be about half of those set forth above.

In one embodiment, such compositions comprise skeletal muscle cells and fibroblast cells and can comprise from about 20% to about 70% myoblasts and, preferably, from about 40-60% myoblasts or about 50% myoblasts. In another embodiment the composition comprises at least about 20%, 25%, 30%, 35%, 40%, 45%, 50%, 55%, 60%, 65%, 70%, 75%, 80%, 85%, 90%, 95%, 96%, 97%, 98% or 99% myoblasts. Compositions having these percentages of myoblasts can be prepared, e.g., using standard cell sorting techniques to obtain purified populations of cells. The purified populations of cells can then be mixed to obtain compositions comprising the desired percentage of myoblasts. Alternatively, compositions comprising the desired percentage of myoblasts can be obtained by culturing a freshly isolated population of skeletal myoblasts in vitro for a limited number of population doublings such that the percentage of myoblasts in the composition falls within the desired range. While not wishing to be bound by any theory, we note that it is possible that presence of fibroblasts within a transplantable composition of the invention may enhance myoblast survival, proliferation, or differentiation and/or graft strength, new vessel formation, etc. Thus it may be desirable to include varying percentages of fibroblasts within the transplantable compositions of the invention.

In yet another embodiment, muscle cells can be combined with fibroblasts derived from a tissue source other than muscle tissue, e.g., with fibroblasts from derived from a different tissue source than the muscle cells of the invention, e.g., skin.

The relative percentage of myoblasts and fibroblasts in a composition can be determined, e.g., by staining one or both populations of cells with a cell specific marker and determining the percentage of cells in the composition which express the marker, e.g., using standard techniques such as FACS analysis. For example, an antibody that recognizes a marker present on either myocytes or fibroblasts can be used to detect one or the other or both cell types to thereby determine the relative percentage of each cell type. For example, when an antibody that recognizes myoblasts is used, the percentage of myoblasts in a composition is determined by assessing the percentage of cells which stain with the antibody and the percentage of fibroblasts is determined by subtracting the percentage of myoblasts from 100. In one embodiment, an antibody that recognizes an α7β1 integrin or which recognizes myosin heavy chain present on or in myocytes can be used (Schweitzer et al. 1987. Experimental Cell Research. 172:1). If an internal marker is used, the cells can be permeabilized prior to staining. A primary antibody used for staining can be directly labeled and used for staining or a secondary antibody can be used to detect binding of the primary antibody to cells.

Cells and compositions of the invention can be used fresh, or can be cultured and/or cryopreserved prior to their use in transplantation. Standard methods for cryopreservation may be used.

III. Preparation of Cells for Transplantation

The cells of the invention can be expanded in vitro prior to transplantation. In one embodiment, the present invention features a population (i.e., a group of two or more cells) of muscle cells for use in transplantation. The muscle cells of the invention can be grown as a cell culture, i.e., as a population of cells which grow in vitro, in a medium suitable to support the growth of the cells prior to administration to a subject.

Media which can be used to support the growth and/or viability of muscle cells are known in the art and include mammalian cell culture media, such as those produced by Gibco BRL (Gaithersburg, Md.). See 1994 Gibco BRL Catalogue & Reference Guide. The medium can be serum-free but is preferably supplemented with animal serum such as fetal calf serum. Optionally, growth factors can be included. Media which are used to promote proliferation of muscle cells and media which are used for maintenance of cells prior to transplantation can differ. A preferred growth medium for the muscle cells is MCDB 120+dexamethasone, e.g., 0.39 μg/ml,+Epidermal Growth Factor (EGF), e.g., 10 ng/ml,+fetal calf serum, e.g., 15%. A preferred medium for muscle cell maintenance is DMEM supplemented with protein, e.g., 10% horse serum. Other exemplary media are taught, for example, in Henry et al. 1995. Diabetes. 44:936; WO 98/54301; and Li et al. 1998. Can. J. Cardiol. 14:735).

In one embodiment, skeletal myoblast cells can be seeded on laminin coated plates for expansion in myoblast growth Basal Medium containing 10% FBS, dexamethasone and EGF. Myoblast enriched plates are expanded for 48 hours and harvested for transplantation. Cells can be harvested using 0.05% trypsin-EDTA and washed in medium containing FBS. These isolations may contain 30 to 50% myoblasts as verified by myotube fusion formation and flow cytometry using a myoblast or fibroblast specific monoclonal antibody. According to certain embodiments of the invention the harvested cell populations may contain approximately 50% to 60% myoblasts. According to certain other embodiments of the invention the harvested cell populations may contain approximately 60% to 75% myoblasts. According to certain other embodiments of the invention the harvested cell populations may contain approximately 75% to 90% myoblasts. According to certain other embodiments of the invention the harvested cell populations may contain approximately 90% to 95% myoblasts. According to certain other embodiments of the invention the harvested cell populations may contain approximately 95% to 99% myoblasts. According to certain other embodiments of the invention the harvested cell populations may contain greater than 99% myoblasts. Where the percentage of myoblasts in the harvested cell population differs from that desired for the transplantable composition, the percentages may be adjusted by cell sorting and/or by combining different cell populations as described above.

According to certain embodiments of the invention the cells are expanded in culture under conditions selected to minimize or reduce the likelihood of myoblast fusion. For example, it may be desirable to maintain the cells in a subconfluent state. It may be desirable to maintain the cells under conditions of less than approximately 50% confluence, of less than approximately 50% to 75% confluence, or of less than approximately 75% to 90% confluence. To ensure that cells do not exceed desired confluence, they may be passaged at appropriate intervals.

When cardiomyocytes are grown in culture, preferably at least about 20%, more preferably at least about 30%, yet more preferably at least about 40%, still more preferably at least about 50%, and most preferably at least about 60% or more of the cardiomyocytes express cardiac troponin and/or myosin, among other cardiac-specific cell products.

In one embodiment, muscle cells of the invention are cultured on a surface coated with poly L lysine and laminin in a medium comprising EGF. The surface coated can alternatively be coated with collagen with a medium comprising FGF. The surface can be a petri dish or a surface suitable for large scale culture of cells. The culture time in vitro is a maximum of about 14 days and is preferably about 7 days. The cells can be permitted to double population about one time in vitro up to about 10 times in vitro. Preferably, the cells are permitted to double population about 5 times in vitro. Preferably, the cells are permitted to double population up to about 10 times such that the fibroblast to myoblast ratio is approximately 1:2 to 1:1.

IV. Modification of Cells

The invention also provides for altering an antigen on the surface of a cell by modifying, masking, or eliminating an antigen on the surface of a cell in the composition is such that upon transplantation of the composition into a subject lysis of the cell is inhibited. Preferably, the antigen is masked with an antibody or a fragment or derivative thereof that binds to the antigen, more preferably the antibody is a monoclonal antibody, and even more preferably the antibody is an anti-MHC class I antibody or a fragment thereof. Preferably, the fragment is a F(ab′)2 fragment. Such masking, modifying or eliminating is preferably done to allogeneic cells or stem cells.

In an unmodified or unaltered state, the antigen on the cell surface stimulates an immune response against the cell (also referred to herein as the donor cell) when the cell is administered to a subject (also referred to herein as the recipient, host, or recipient subject). By altering the antigen, the normal immunological recognition of the donor cell by the immune system cells of the recipient is disrupted and additionally, “abnormal” immunological recognition of this altered form of the antigen can lead to donor cell-specific long term unresponsiveness in the recipient. Thus, alteration of an antigen on the donor cell prior to administering the cell to a recipient interferes with the initial phase of recognition of the donor cell by the cells of the host's immune system subsequent to administration of the cell. Furthermore, alteration of the antigen can induce immunological nonresponsiveness or tolerance, thereby preventing the induction of the effector phases of an immune response (e.g., cytotoxic T cell generation, antibody production etc.) which are ultimately responsible for rejection of foreign cells in a normal immune response. As used herein, the terms “altered” and “modified” are used interchangeably and encompass changes that are made to a donor cell antigen which reduce the immunogenicity of the antigen to thereby interfere with immunological recognition of the antigen by the recipient's immune system. Preferably immunological nonresponsiveness to the donor cells in the recipient subject is generated as a result of alteration of the antigen. The terms “altered” and “modified” are not intended to include complete elimination of the antigen on the donor cell since delivery of an inappropriate or insufficient signal to the host's immune cells may be necessary to achieve immunological nonresponsiveness.

Antigens to be altered according to the invention include antigens on a donor cell which can interact with an immune cell (e.g., a hematopoietic cell, an NK cell, an LAK cell) in an allogeneic or xenogeneic recipient and thereby stimulate a specific immune response against the donor cell in the recipient. The interaction between the antigen and the immune cell may be an indirect interaction (e.g., mediated by soluble factors which induce a response in the hematopoietic cell, e.g., humoral mediated) or, preferably, is a direct interaction between the antigen and a molecule present on the surface of the immune cell (i.e., cell-cell mediated). As used herein, the phrase “immune cell” is intended to include hematopoietic cells such as T lymphocytes, B lymphocytes, monocytes, macrophages, dendritic cells, and other antigen presenting cells, NK cells, and LAK cells. In preferred embodiments, the antigen is one which interacts with a T lymphocyte in the recipient (e.g., the antigen normally binds to a receptor on the surface of a T lymphocyte), or with an NK cell or LAK cell in the recipient.

In a preferred embodiment, the antigen on the donor cell to be altered is an MHC class I antigen. MHC class I antigens are present on almost all cell types. In a normal immune response, self MHC molecules function to present antigenic peptides to a T cell receptor (TCR) on the surface of self T lymphocytes. In immune recognition of allogeneic or xenogeneic cells, foreign MHC antigens (most likely together with a peptide bound thereto) on donor cells are recognized by the T cell receptor on host T cells to elicit an immune response. In addition, foreign MHC class I antigens are known to be recognized by MHC class I receptors on NK cells. MHC class I antigens on a donor cell are altered to interfere with their recognition by T cells, NK cells, or LAK cells in an allogeneic or xenogeneic host (e.g., a portion of the MHC class I antigen which is normally recognized by the T cell receptor, NK cells, or LAK cells is blocked or “masked” such that normal recognition of the MHC class I antigen can no longer occur). Additionally, an altered form of an MHC class I antigen which is exposed to host T cells, NK cells or LAK cells (i.e., available for presentation to the host cell receptor) may deliver an inappropriate or insufficient signal to the host T cell such that, rather than stimulating an immune response against the allogeneic or xenogeneic cell, donor cell-specific T cell non-responsiveness, inhibition of NK-mediated cell rejection, and/or inhibition of LAK-mediated cell rejection is induced. For example, it is known that T cells which receive an inappropriate or insufficient signal through their T cell receptor (e.g., by binding to an MHC antigen in the absence of a costimulatory signal, such as that provided by B7) become anergic rather than activated and can remain refractory to restimulation for long periods of time (see, e.g., Damle et al. (1981) Proc. Natl. Acad. Sci. USA 78:5096-5100; Lesslauer et al. (1986) Eur. J. Immunol. 16:1289-1295; Gimmi, et al. (1991) Proc. Natl. Acad. Sci. USA 88: 6575-6579; Linsley et al. (1991) J. Exp. Med. 173:721-730; Koulova et al. (1991) J. Exp. Med. 173:759-762; Razi-Wolf, et al. (1992) Proc. Natl. Acad. Sci. USA 89:4210-4214).

Alternative to MHC class I antigens, the antigen to be altered on a donor cell can be an MHC class II antigen. Similar to MHC class I antigens, MHC class II antigens function to present antigenic peptides to a T cell receptor on T lymphocytes. However, MHC class II antigens are present on a limited number of cell types (primarily B cells, macrophages, dendritic cells, Langerhans cells and thymic epithelial cells). In addition to or alternative to MHC antigens, other antigens on a donor cell which interact with molecules on host T cells or NK cells and which are known to be involved in immunological rejection of allogeneic or xenogeneic cells can be altered. Other donor cell antigens known to interact with host T cells and contribute to rejection of a donor cell include molecules which function to increase the avidity of the interaction between a donor cell and a host T cell. Due to this property, these molecules are typically referred to as adhesion molecules (although they may serve other functions in addition to increasing the adhesion between a donor cell and a host T cell). Examples of preferred adhesion molecules which can be altered according to the invention include LFA-3 and ICAM-1. These molecules are ligands for the CD2 and LFA-1 receptors, respectively, on T cells. By altering an adhesion molecule on the donor cell, (such as LFA-3, ICAM-1 or a similarly functioning molecule), the ability of the host's T cells to bind to and interact with the donor cell is reduced. Both LFA-3 and ICAM-1 are found on endothelial cells found within blood vessels in transplanted organs such as kidney and heart. Altering these antigens can facilitate transplantation of any vascularized implant, by altering recognition of those antigens by CD2+ and LFA-1+ host T-lymphocytes.

The presence of MHC molecules or adhesion molecules such as LFA-3, ICAM-1 etc. on a particular donor cell can be assessed by standard procedures known in the art. For example, the donor cell can be reacted with a labeled antibody directed against the molecule to be detected (e.g., MHC molecule, ICAM-1, LFA-1 etc.) and the association of the labeled antibody with the cell can be measured by a suitable technique (e.g., immunohistochemistry, flow cytometry etc.).

A preferred method for altering an antigen on a donor cell to inhibit an immune response against the cell is to contact the cell with a molecule which binds to the antigen on the cell surface. It is preferred that the cell be contacted with the molecule which binds to the antigen prior to administering the cell to a recipient (i.e., the cell is contacted with the molecule in vitro). For example, the cell can be incubated with the molecule which binds the antigen under conditions which allow binding of the molecule to the antigen and then any unbound molecule can be removed. Following administration of the modified cell to a recipient, the molecule remains bound to the antigen on the cell for a sufficient time to interfere with immunological recognition by host cells and induce non-responsiveness in the recipient.

Preferably, the molecule for binding to an antigen on a donor cell is an antibody, or fragment or derivative thereof which retains the ability to bind to the antigen. For use in therapeutic applications, it is necessary that the antibody which binds the antigen to be altered be unable to fix complement, thus preventing donor cell lysis. Antibody complement fixation can be prevented by deletion of an Fc portion of an antibody, by using an antibody isotype which is not capable of fixing complement, or by using a complement fixing antibody in conjunction with a drug which inhibits complement fixation. Alternatively, amino acid residues within the Fc region which are necessary for activating complement (see e.g., Tan et al. (1990) Proc. Natl. Acad. Sci. USA 87:162-166; Duncan and Winter (1988) Nature 332: 738-740) can be mutated to reduce or eliminate the complement-activating ability of an intact antibody. Likewise, amino acids residues within the Fc region which are necessary for binding of the Fc region to Fc receptors (see e.g., Canfield, S. M. and S. L. Morrison (1991) J. Exp. Med. 173:1483-1491; and Lund, J. et al. (1991) J. Immunol. 147:2657-2662) can also be mutated to reduce or eliminate Fc receptor binding if an intact antibody is to be used.

A preferred antibody fragment for altering an antigen is an F(ab′)₂ fragment. Antibodies can be fragmented using conventional techniques. For example, the Fc portion of an antibody can be removed by treating an intact antibody with pepsin, thereby generating an F(ab′)₂ fragment. In a standard procedure for generating F(ab′)₂ fragments, intact antibodies are incubated with immobilized pepsin and the digested antibody mixture is applied to an immobilized protein A column. The free Fc portion binds to the column while the F(ab′)₂ fragments passes through the column. The F(ab′)₂ fragments can be further purified by HPLC or FPLC. F(ab′)₂ fragments can be treated to reduce disulfide bridges to produce Fab′ fragments.

An antibody, or fragment or derivative thereof, to be used to alter an antigen can be derived from polygonal antisera containing antibodies reactive with a number of epitopes on an antigen. Preferably, the antibody is a monoclonal antibody directed against the antigen. Polyclonal and monoclonal antibodies can be prepared by standard techniques known in the art. For example, a mammal, (e.g., a mouse, hamster, or rabbit) can be immunized with the antigen or with a cell which expresses the antigen (e.g., on the cell surface) to elicit an antibody response against the antigen in the mammal. Alternatively, tissue or a whole organ which expresses the antigen can be used to elicit antibodies. The progress of immunization can be monitored by detection of antibody titers in plasma or serum. Standard ELISA or other immunoassay can be used with the antigen to assess the levels of antibodies. Following immunization, antisera can be obtained and, if desired, polyclonal antibodies isolated from the sera. To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art. For example, the hybridoma technique originally developed by Kohler and Milstein ((1975) Nature 256:495-497) as well as other techniques such as the human B-cell hybridoma technique (Kozbar et al., (1983) Immunol. Today 4:72), and the EBV-hybridoma technique to produce human monoclonal antibodies (Cole et al. (1985) Monoclonal Antibodies in Cancer Therapy, Allen R. Bliss, Inc., pages 77-96) can be used. Hybridoma cells can be screened immunochemically for production of antibodies specifically reactive with the antigen and monoclonal antibodies isolated.

Another method of generating specific antibodies, or antibody fragments, reactive against the antigen is to screen expression libraries encoding immunoglobulin genes, or portions thereof, expressed in bacteria with the antigen (or a portion thereof). For example, complete Fab fragments, V_{H} regions, F_{V} regions and single chain antibodies can be expressed in bacteria using phage expression libraries. See e.g., Ward et al., (1989) Nature 341:544-546; Huse et al., (1989) Science 246:1275-1281; and McCafferty et al. (1990) Nature 348:552-554. Alternatively, a SCID-hu mouse can be used to produce antibodies, or fragments thereof (available from Genpharm). Antibodies of the appropriate binding specificity which are made by these techniques can be used to alter an antigen on a donor cell.

An antibody, or fragment thereof, produced in a non-human subject can be recognized to varying degrees as foreign when the antibody is administered to a human subject (e.g., when a donor cell with an antibody bound thereto is administered to a human subject) and an immune response against the antibody may be generated in the subject. One approach for minimizing or eliminating this problem is to produce chimeric or humanized antibody derivatives, i.e., antibody molecules comprising portions which are derived from nonhuman antibodies and portions which are derived from human antibodies. Chimeric antibody molecules can include, for example, an antigen binding domain from an antibody of a mouse, rat, or other species, with human constant regions. A variety of approaches for making chimeric antibodies have been described. See e.g., Morrison et al., Proc. Natl. Acad. Sci. U.S.A. 81, 6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B. For use in therapeutic applications, it is preferred that an antibody used to alter a donor cell antigen not contain an Fc portion. Thus, a humanized F(ab′)₂ fragment in which parts of the variable region of the antibody, especially the conserved framework regions of the antigen-binding domain, are of human origin and only the hypervariable regions are of non-human origin is a preferred antibody derivative. Such altered immunoglobulin molecules can be made by any of several techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and are preferably made according to the teachings of PCT Publication WO92/06193 or EP 0239400. Humanized antibodies can be commercially produced by, for example, Scotgen Limited, 2 Holly Road, Twickenham, Middlesex, Great Britain.

Each of the cell surface antigens to be altered, e.g., MHC class I antigens, MHC class II antigens, LFA-3 and ICAM-1 are well-characterized molecules and antibodies to these antigens are commercially available. For example, an antibody directed against human MHC class I antigens (i.e., an anti-HLA class I antibody), W6/32, is available from the American Type Culture Collection (ATCC HB 95). This antibody was raised against human tonsillar lymphocyte membranes and binds to HLA-A, HLA-B and HLA-C (Barnstable, C. J. et al. (1978) Cell 14:9-20). Another anti-MHC class I antibody which can be used is PT85 (see Davis, W. C. et al. (1984) Hybridoma Technology in Agricultural and Veterinary Research. N. J. Stern and H. R. Gamble, eds., Rownman and Allenheld Publishers, Totowa, N.J., p121; commercially available from Veterinary Medicine Research Development, Pullman, Wash.). This antibody was raised against swine leukocyte antigens (SLA) and binds to class I antigens from several different species (e.g., pig, human, mouse, goat). An anti-ICAM-1 antibody can be obtained from AMAC, Inc., Maine. Hybridoma cells producing anti-LFA-3 can be obtained from the American Type Culture Collection, Rockville, Md. In a preferred embodiment, the antibody is PT85.

A suitable antibody, or fragment or derivative thereof, for use in the invention can be identified based upon its ability to inhibit the immunological rejection of allogeneic or xenogeneic cells. Briefly, the antibody (or antibody fragment) is incubated for a short period of time (e.g., 30 minutes at room temperature) with cells or tissue to be transplanted and any unbound antibody is washed away. The cells or tissue are then transplanted into a recipient animal. The ability of the antibody pretreatment to inhibit or prevent rejection of the transplanted cells or tissue is then determined by monitoring for rejection of the cells or tissue compared to untreated controls.

It is preferred that an antibody, or fragment or derivative thereof, which is used to alter an antigen have an affinity for binding to the antigen of at least 10⁻⁷ M. The affinity of an antibody or other molecule for binding to an antigen can be determined by conventional techniques (see Masan, D. W. and Williams, A. F. (1980) Biochem. J. 187:1-10). Briefly, the antibody to be tested is labeled with ¹²⁵I and incubated with cells expressing the antigen at increasing concentrations until equilibrium is reached. Data are plotted graphically as [bound antibody]/[free antibody] versus [bound antibody] and the slope of the line is equal to the kD (Scatchard analysis).

Other molecules which bind to an antigen on a donor cell and produce a functionally similar result as antibodies, or fragments or derivatives thereof, (e.g., other molecules which interfere with the interaction of the antigen with a hematopoietic cell and induce immunological nonresponsiveness) can be used to alter the antigen on the donor cell. One such molecule is a soluble form of a ligand for an antigen (e.g., a receptor) on the donor cell which could be used to alter the antigen on the donor cell. For example, a soluble form of CD2 (i.e., comprising the extracellular domain of CD2 without the transmembrane or cytoplasmic domain) can be used to alter LFA-3 on the donor cell by binding to LFA-3 on donor cells in a manner analogous to an antibody. Alternatively, a soluble form of LFA-1 can be used to alter ICAM-1 on the donor cell. A soluble form of a ligand can be made by standard recombinant DNA procedures, using a recombinant expression vector containing DNA encoding the ligand encompassing an extracellular domain (i.e., lacking DNA encoding the transmembrane and cytoplasmic domains). The recombinant expression vector encoding the extracellular domain of the ligand can be introduced into host cells to produce a soluble ligand, which can then be isolated. Soluble ligands of use have a binding affinity for the receptor on the donor cell sufficient to remain bound to the receptor to interfere with immunological recognition and induce non-responsiveness when the cell is administered to a recipient (e.g., preferably, the affinity for binding of the soluble ligand to the receptor is at least about 10⁻⁷ M). Additionally, the soluble ligand can be in the form of a fusion protein comprising the receptor binding portion of the ligand fused to another protein or portion of a protein. For example, an immunoglobulin fusion protein which includes an extracellular domain, or functional portion of CD2 or LFA-1 linked to an immunoglobulin heavy chain constant region (e.g., the hinge, CH2 and CH3 regions of a human immunoglobulin such as IgG1) can be used. Immunoglobulin fusion proteins can be prepared, for example, according to the teachings of Capon, D. J. et al. (1989) Nature 337:525-531 and U.S. Pat. No. 5,116,964 to Capon and Lasky.

Another type of molecule which can be used to alter an MHC antigen (e.g., and MHC class I antigen) is a peptide which binds to the MHC antigen and interferes with the interaction of the MHC antigen with a T lymphocyte, NK cell, or LAK cell. In one embodiment, the soluble peptide mimics a region of the T cell receptor which contacts the MHC antigen. This peptide can be used to interfere with the interaction of the intact T cell receptor (on a T lymphocyte) with the MHC antigen. Such a peptide binds to a region of the MHC molecule which is specifically recognized by a portion of the T cell receptor (e.g., the alpha-1 or alpha-2 domain of an MHC class I antigen), thereby altering the MHC class I antigen and inhibiting recognition of the antigen by the T cell receptor. In another embodiment, the soluble peptide mimics a region of a T cell surface molecule which contacts the MHC antigen, such as a region of the CD8 molecule which contacts an MHC class I antigen or a region of a CD4 molecule which contacts an MHC class II antigen. For example, a peptide which binds to a region of the alpha-3 loop of an MHC class I antigen can be used to inhibit binding to CD8 to the antigen, thereby inhibiting recognition of the antigen by T cells. T cell receptor-derived peptides have been used to inhibit MHC class I-restricted immune responses (see e.g., Clayberger, C. et al. (1993) Transplant Proc. 25:477-478) and prolong allogeneic skin graft survival in vivo when injected subcutaneously into the recipient (see e.g., Goss, J. A. et al. (1993) Proc. Natl. Acad. Sci. USA 90:9872-9876).

An antigen on a donor cell further can be altered by using two or more molecules which bind to the same or different antigen. For example, two different antibodies with specificity for two different epitopes on the same antigen can be used (e.g., two different anti-MHC class I antibodies can be used in combination). Alternatively, two different types of molecules which bind to the same antigen can be used (e.g., an anti-MHC class I antibody and an MHC class I-binding peptide). A preferred combination of anti-MHC class I antibodies which can be used with human cells is the W6/32 antibody and the PT85 antibody or F(ab′)₂ fragments thereof. When the donor cell to be administered to a subject bears more than one hematopoietic cell-interactive antigen, two or more treatments can be used together. For example, two antibodies, each directed against a different antigen (eg., an anti-MHC class I antibody and an anti-ICAM-1 antibody) can be used in combination or two different types of molecules, each binding to a different antigen, can be used (e.g., an anti-ICAM-1 antibody and an MHC class I-binding peptide). Alternatively, polyclonal antisera generated against the entire donor cell or tissue containing donor cells can be used, following removal of the Fc region, to alter multiple cell surface antigens of the donor cells.

The ability of two different monoclonal antibodies which bind to the same antigen to bind to different epitopes on the antigen can be determined using a competition binding assay. Briefly, one monoclonal antibody is labeled and used to stain cells which express the antigen. The ability of the unlabeled second monoclonal antibody to inhibit the binding of the first labeled monoclonal antibody to the antigen on the cells is then assessed. If the second monoclonal antibody binds to a different epitope on the antigen than does the first antibody, the second antibody will be unable to competitively inhibit the binding of the first antibody to the antigen.

A preferred method for altering at least two different epitopes on an antigen on a donor cell to inhibit an immune response against the cell is to contact the cell with at least two different molecules which bind to the epitopes. It is preferred that the cell be contacted with at least two different molecules which bind to the different epitopes prior to administering the cell to a recipient (i.e., the cell is contacted with the molecule in vitro). For example, the cell can be incubated with the molecules which bind to the epitopes under conditions which allow binding of the molecules to the epitopes and then any unbound molecules can be removed. Following administration of the donor cell to a recipient, the molecules remain bound to the epitopes on the surface antigen for a sufficient time to interfere with immunological recognition by host cells and induce non-responsiveness in the recipient.

Alternative to binding a molecule (e.g., an antibody) to an antigen on a donor cell to inhibit immunological rejection of the cell, the antigen on the donor cell can be altered by other means. For example, the antigen can be directly altered (e.g., mutated) such that it can no longer interact normally with an immune cell, e.g., a T lymphocyte), an NK cell, or an LAK cell, in an allogeneic or xenogeneic recipient and induces immunological non-responsiveness to the donor cell in the recipient. For example, a mutated form of a class I MHC antigen or adhesion molecule (e.g., LFA-3 or ICAM-1) which does not contribute to T cell activation but rather delivers an inappropriate or insufficient signal to a T cell upon binding to a receptor on the T cell can be created by mutagenesis and selection. A nucleic acid encoding the mutated form of the antigen can then be inserted into the genome of a non-human animal, either as a transgene or by homologous recombination (to replace the endogenous gene encoding the wild-type antigen). Cells from the non-human animal which express the mutated form of the antigen can then be used as donor cells for transplantation into an allogeneic or xenogeneic recipient.

Alternatively, an antigen on the donor cell can be altered by downmodulating or altering its level of expression on the surface of the donor cell such that the interaction between the antigen and a recipient immune cell is modified. By decreasing the level of surface expression of one or more antigens on the donor cell, the avidity of the interaction between the donor cell and the immune cell e.g., T lymphocyte, NK cell, LAK cell, is reduced. The level of surface expression of an antigen on the donor cell can be down-modulated by inhibiting the transcription, translation or transport of the antigen to the cell surface. Agents which decrease surface expression of the antigen can be contacted with the donor cell. For example, a number of oncogenic viruses have been demonstrated to decrease MHC class I expression in infected cells (see e.g., Travers et al. (1980) Int'l. Symp. on Aging in Cancer, 175180; Rees et al. (1988) Br. J. Cancer, 57:374-377). In addition, it has been found that this effect on MHC class I expression can be achieved using fragments of viral genomes, in addition to intact virus. For example, transfection of cultured kidney cells with fragments of adenovirus causes elimination of surface MHC class I antigenic expression (Whoshi et al. (1988) J. Exp. Med. 168:2153-2164). For purposes of decreasing MHC class I expression on the surfaces of donor cells, viral fragments which are non-infectious are preferable to whole viruses.

Alternatively, the level of an antigen on the donor cell surface can be altered by capping the antigen. Capping is a term referring to the use of antibodies to cause aggregation and inactivation of surface antigens. To induce capping, a tissue is contacted with a first antibody specific for an antigen to be altered, to allow formation of antigen-antibody immune complexes. Subsequently, the tissue is contacted with a second antibody which forms immune complexes with the first antibody. As a result of treatment with the second antibody, the first antibody is aggregated to form a cap at a single location on the cell surface. The technique of capping is well known and has been described, e.g., in Taylor et al. (1971), Nat. New Biol. 233:225-227; and Santiso et al. (1986), Blood, 67:343-349. To alter MHC class I antigens, donor cells are incubated with a first antibody (e.g., W6/32 antibody, PT85 antibody) reactive with MHC class I molecules, followed by incubation with a second antibody reactive with the donor species, e.g., goat anti-mouse antibody, to result in aggregation.

V. Genetic Modification of Cells

Muscle cells of the invention (or other cells included in the muscle cell compositions of the invention) can be “modified to express a gene product”. As used herein, the term “modified to express a gene product” is intended to mean that the cell is treated in a manner that results in the production of a gene product by the cell. Preferably, the cell does not express the gene product prior to modification. Alternatively, modification of the cell may result in an increased production of a gene product already expressed by the cell or result in production of a gene product (e.g., an antisense RNA molecule) which decreases production of another, undesirable gene product normally expressed by the cell.

In one embodiment, skeletal muscle cells are modified to produce a gene product that makes them more cardiac-like, e.g., connexin43 (J. Cell. Biol. 1989. 108:595).

In a preferred embodiment, a cell is modified to express a gene product by introducing genetic material, such as a nucleic acid molecule (e.g., RNA or, more preferably, DNA) into the cell. The nucleic acid molecule introduced into the cell encodes a gene product to be expressed by the cell. The term “gene product” as used herein is intended to include proteins, peptides and functional RNA molecules. Generally, the gene product encoded by the nucleic acid molecule is the desired gene product to be supplied to a subject. Alternatively, the encoded gene product is one which induces the expression of the desired gene product by the cell (e.g., the introduced genetic material encodes a transcription factor which induces the transcription of the gene product to be supplied to the subject).

Examples of gene products that can be delivered to a subject via a genetically modified muscle cells include gene products that can prevent future cardiac disorders, such as growth factors which encourage blood vessels to invade the heart muscle, e.g., Fibroblast Growth Factor (FGF) 1, FGF-2, Transforming Growth Factor β (TGF-β), and angiotensin. Other gene products that can be delivered to a subject via a genetically modified cardiomyocyte include factors which promote cardiomyocyte survival, such as FGF, TGF-β, IL-10, CTLA 4-Ig, and bcl-2.

A nucleic acid molecule introduced into a cell is in a form suitable for expression in the cell of the gene product encoded by the nucleic acid. Accordingly, the nucleic acid molecule includes coding and regulatory sequences required for transcription of a gene (or portion thereof) and, when the gene product is a protein or peptide, translation of the gene product encoded by the gene. Regulatory sequences which can be included in the nucleic acid molecule include promoters, enhancers and polyadenylation signals, as well as sequences necessary for transport of an encoded protein or peptide, for example N-terminal signal sequences for transport of proteins or peptides to the surface of the cell or for secretion.

Nucleotide sequences which regulate expression of a gene product (e.g., promoter and enhancer sequences) are selected based upon the type of cell in which the gene product is to be expressed and the desired level of expression of the gene product. For example, a promoter known to confer cell-type specific expression of a gene linked to the promoter can be used. A promoter specific for myoblast gene expression can be linked to a gene of interest to confer muscle-specific expression of that gene product. Muscle-specific regulatory elements which are known in the art include upstream regions from the dystrophin gene (Klamut et al., (1989) Mol. Cell. Biol. 9:2396), the creatine kinase gene (Buskin and Hauschka, (1989) Mol. Cell Biol. 9:2627) and the troponin gene (Mar and Ordahl, (1988) Proc. Natl. Acad. Sci. USA. 85:6404). Regulatory elements specific for other cell types are known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters). Alternatively, a regulatory element which can direct constitutive expression of a gene in a variety of different cell types, such as a viral regulatory element, can be used. Examples of viral promoters commonly used to drive gene expression include those derived from polyoma virus, Adenovirus 2, cytomegalovirus and Simian Virus 40, and retroviral LTRs. Alternatively, a regulatory element which provides inducible expression of a gene linked thereto can be used. The use of an inducible regulatory element (e.g., an inducible promoter) allows for modulation of the production of the gene product in the cell. Examples of potentially useful inducible regulatory systems for use in eukaryotic cells include hormone-regulated elements (e.g., see Mader, S. and White, J. H. (1993) Proc. Natl. Acad. Sci. USA 90:5603-5607), synthetic ligand-regulated elements (see, e.g. Spencer, D. M. et al. (1993) Science 262:1019-1024) and ionizing radiation-regulated elements (e.g., see Manome, Y. et al. (1993) Biochemistry 32:10607-10613; Datta, R. et al. (1992) Proc. Natl. Acad. Sci. USA 89:10149-10153). Additional tissue-specific or inducible regulatory systems which may be developed can also be used in accordance with the invention.

There are a number of techniques known in the art for introducing genetic material into a cell that can be applied to modify a cell of the invention. In one embodiment, the nucleic acid is in the form of a naked nucleic acid molecule. In this situation, the nucleic acid molecule introduced into a cell to be modified consists only of the nucleic acid encoding the gene product and the necessary regulatory elements. Alternatively, the nucleic acid encoding the gene product (including the necessary regulatory elements) is contained within a plasmid vector. Examples of plasmid expression vectors include CDM8 (Seed, B. (1987) Nature 329:840) and pMT2PC (Kaufman, et al. (1987) EMBO J. 6:187-195). In another embodiment, the nucleic acid molecule to be introduced into a cell is contained within a viral vector. In this situation, the nucleic acid encoding the gene product is inserted into the viral genome (or a partial viral genome). The regulatory elements directing the expression of the gene product can be included with the nucleic acid inserted into the viral genome (i.e., linked to the gene inserted into the viral genome) or can be provided by the viral genome itself.

Naked DNA can be introduced into cells by forming a precipitate containing the DNA and calcium phosphate. Alternatively, naked DNA can also be introduced into cells by forming a mixture of the DNA and DEAE-dextran and incubating the mixture with the cells. or by incubating the cells and the DNA together in an appropriate buffer and subjecting the cells to a high-voltage electric pulse (i.e., by electroporation). A further method for introducing naked DNA cells is by mixing the DNA with a liposome suspension containing cationic lipids. The DNA/liposome complex is then incubated with cells. Naked DNA can also be directly injected into cells by, for example, microinjection. For an in vitro culture of cells, DNA can be introduced by microinjection in vitro or by a gene gun in vivo. Alternatively, naked DNA can also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. An alternative method for generating a cell that is modified to express a gene product involving introducing naked DNA into cells is to create a transgenic animal which contains cells modified to express the gene product of interest.

Use of viral vectors containing nucleic acid, e.g., a cDNA encoding a gene product, is a preferred approach for introducing nucleic acid into a cell. Infection of cells with a viral vector has the advantage that a large proportion of cells receive the nucleic acid, which can obviate the need for selection of cells which have received the nucleic acid. Additionally, molecules encoded within the viral vector, e.g., by a cDNA contained in the viral vector, are expressed efficiently in cells which have taken up viral vector nucleic acid and viral vector systems can be used either in vitro or in vivo.

Defective retroviruses are well characterized for use in gene transfer for gene therapy purposes (for a review see Miller, A. D. (1990) Blood 76:271). A recombinant retrovirus can be constructed having a nucleic acid encoding a gene product of interest inserted into the retroviral genome. Additionally, portions of the retroviral genome can be removed to render the retrovirus replication defective. The replication defective retrovirus is then packaged into virions which can be used to infect a target cell through the use of a helper virus by standard techniques.

The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

When the method used to introduce nucleic acid into a population of cells results in modification of a large proportion of the cells and efficient expression of the gene product by the cells (e.g., as is often the case when using a viral expression vector), the modified population of cells may be used without further isolation or subcloning of individual cells within the population. That is, there may be sufficient production of the gene product by the population of cells such that no further cell isolation is needed. Alternatively, it may be desirable to grow a homogenous population of identically modified cells from a single modified cell to isolate cells which efficiently express the gene product. Such a population of uniform cells can be prepared by isolating a single modified cell by limiting dilution cloning followed by expanding the single cell in culture into a clonal population of cells by standard techniques.

Alternative to introducing a nucleic acid molecule into a cell to modify the cell to express a gene product, a cell can be modified by inducing or increasing the level of expression of the gene product by a cell. For example, a cell may be capable of expressing a particular gene product but fails to do so without additional treatment of the cell. Similarly, the cell may express insufficient amounts of the gene product for the desired purpose. Thus, an agent which stimulates expression of a gene product can be used to induce or increase expression of a gene product by the cell. For example, cells can be contacted with an agent ill vitro in a culture medium. The agent which stimulates expression of a gene product may function, for instance, by increasing transcription of the gene encoding the product, by increasing the rate of translation or stability (e.g., a post transcriptional modification such as a poly A tail) of an mRNA encoding the product or by increasing stability, transport or localization of the gene product. Examples of agents which can be used to induce expression of a gene product include cytokines and growth factors.

Another type of agent which can be used to induce or increase expression of a gene product by a cell is a transcription factor which upregulates transcription of the gene encoding the product. A transcription factor which upregulates the expression of a gene encoding a gene product of interest can be provided to a cell, for example, by introducing into the cell a nucleic acid molecule encoding the transcription factor. Thus, this approach represents an alternative type of nucleic acid molecule which can be introduced into the cell (for example by one of the previously discussed methods). In this case, the introduced nucleic acid does not directly encode the gene product of interest but rather causes production of the gene product by the cell indirectly by inducing expression of the gene product.

In one embodiment, the invention provides a method for promoting a cardiac cell phenotype in a skeletal myoblast by recombinantly expressing a cardiac cell gene product in the myoblast so that the cardiac cell phenotype is promoted. In an embodiment, the gene product is a GATA transcription factor and, preferably is GATA4 or GATA6. The nucleotide sequence encoding GATA6 can be found, e.g., in any public or private database. The sequence is available, e.g., on GenBank as accession number 005257. The sequence is also taught, e.g., in Genomics. 1996. 38(3):283-90. The nucleotide sequence encoding GATA-4 is also available through a variety of databases, e.g., at GenBank accession number L34357. In another embodiment, the cells can be engineered to recombinantly express an angiogenic gene product, such as, CTGF (J Biochem 1999 July 1;126:137), VEGF (Jpn J Cancer Res 1999 January;90:93-100), IGR-I, IGF-II, TGF-β1, PDGF β, or an agent that acts indirectly to induce an angiogenic agent, e.g., FGF 4 (Cancer Res 1997 December 15;57(24):5590-7).

VI. Cellular Transplantation, Transplantable Compositions, and Methods of Treatment

The term “subject” is intended to include mammals, particularly humans. Examples of subjects include primates (e.g., humans, and monkeys). Subjects suitable for transplantation using the instant methods having disorders characterized by insufficient cardiac function or cardiac damage or myocardial ischemic damage.

Transplantation of muscle cells of the invention into the heart of a subject with cardiac dysfunction or damage, e.g., cardiac dysfunction or damage due to myocardial ischemia may improve cardiac function in a variety of ways. Transplantable compositions of the invention may supplement existing cardiomyocytes and/or result in replacement of lost cardiomyocytes. According to certain embodiments of the invention, following delivery to the heart skeletal myoblasts and/or fibroblasts survive, differentiate, and/or proliferate. For example, according to certain embodiments of the invention skeletal myoblasts fuse in vivo to form myotubes and/or myofibers. Evidence of skeletal myoblast survival, differentiation, and/or proliferation, e.g., evidence of myotube and/or myofiber formation may be obtained by examining cardiac tissue for cellular expression of genes and/or proteins (markers) that are characteristic of such cells. Evidence of angiogenesis may be obtained by examining cardiac tissue for cells that express genes and/or proteins characteristic of endothelial cells. In particular, cardiac tissue can be examined for the presence of cells expressing genes and/or proteins that are present in one or more of the following cell types: skeletal myoblasts, skeletal myotubes, skeletal myofibers, fibroblasts, and endothelial cells. A variety of markers that are well known in the art may be used. Although immunohistochemical examination of tissue specimens is a convenient means of assessing protein expression (see Examples), other appropriate means of assessing mRNA or protein expression may be used including, e.g., PCR, microarray hybridization, Northern or Western blots, etc. Skeletal myoblasts may be distinguished from both more differentiated skeletal muscle cells (e.g., myotubes or myofibers) and from cardiac cells by examining such cells for expression of markers such as myogenin, myoD, or myf-5. Since mature heart muscle lacks myoblasts, such markers distinguish introduced myoblasts both from more differentiated skeletal muscle cells and from cells of cardiac origin. To distinguish more differentiated cells of skeletal origin from cardiac cells, expression of a marker characteristic of skeletal muscle such as skeletal muscle-specific myosin may be used. While less conclusive than actual examination of cardiac tissue following transplantation, functional evidence of engraftment and survival may be obtained using any of a variety of methods known in the art. For example, imaging studies may be used to assess ejection fraction, wall motion, cellular metabolism, etc. Clinical evidence of improvement may also be obtained, e.g., by assessing exercise tolerance, symptoms such as dyspnea or chest pain, etc.

As used herein the terms “administering”, “introducing”, “delivering” and “transplanting” are used interchangeably and refer to the placement of the muscle cells of the invention into a subject, e.g., a syngeneic, allogeneic,.or a xenogeneic subject, by a method or route which results in localization of the muscle cells at a desired site, e.g., at the site of cardiac damage in the subject.

In one embodiment the cells of the invention are introduced into a subject having cardiac damage in the left ventricle. In another embodiment, the cells of the invention are introduced into a subject having cardiac damage in the anterior portion of the left ventricle. In another embodiment, the cells of the invention are introduced into a subject having cardiomyopathy, e.g., hypertrophic or dilated in nature. In another embodiment, the cells of the invention are introduced into a subject having myocardial ischemic damage. In yet another embodiment, the cells are administered to a subject having cardiac damage characterized by an ejection fraction of less than 50%, e.g., 40-50%.

The invention further provides methods for treating a condition in a subject characterized by damage to cardiac tissue comprising transplanting a muscle cell or muscle cell composition of the invention into the subject such that the condition is thereby treated. According to certain embodiments of the invention muscle cells are introduced into a subject with a cardiac disorder in an amount sufficient to result in at least partial reduction or alleviation of at least one adverse effect or symptom of the cardiac disorder. According to certain embodiments of the invention, the cells are transplanted into an ischemic zone of the heart. According to certain embodiments of the invention cells are delivered to myocardial scar tissue. According to certain embodiments of the invention cells delivered to tissue in the vicinity of a myocardial scar instead of or in addition to delivery to myocardial scar tissue. In another embodiment, the muscle cells are introduced into a subject in an amount sufficient to replace lost or damaged cardiomyocytes. According to certain embodiments of the invention, the composition is transplanted by direct injection into the damaged or dysfuntional cardiac tissue (e.g., cardiac tissue damaged by ischemia, or into fibrotic tissue or scar tissue). In certain embodiments of the invention, a catheter is used to inject the composition. The cardiac tissue may be damaged or dysfunctional due to any of a number of causes as described above, e.g., an infarction, myocardial ischemic damage or cardiomyopathy, etc. The area to be treated can be located in a ventricle wall. In a preferred method the area to be treated, e.g., the area of cardiac damage, is located in a ventricle wall such as the left ventricle wall. In a preferred embodiment of the invention autologous cells, e.g., cells that have been obtained from the subject and expanded in culture as described herein, are transplanted. In another embodiment, the composition is transplanted into a coronary vessel of the subject.

One method that can be used to deliver the muscle cells of the invention to a subject is direct injection of the muscle cells into the ventricular myocardium of the subject. See e.g., Soonpaa, M. H. et al. (1994) Science 264:98-101; Koh, G. Y. et al. (1993) Am. J. Physiol. 33:H1727-1733. Muscle cells can be administered in a physiologically compatible carrier, such as a buffered saline solution. The number of cells to be administered can vary. The number can be selected based on criteria such as the size of an area of cardiac damage, the functional state of the heart, etc. In addition, factors such as the length of time available for expanding the cells prior to delivery may constrain the number of cells administered. According to certain embodiments of the invention, when treating a human subject between approximately 10⁶ and 10⁹ cells, for example between approximately 10⁶ and 10⁷, 10⁷ and 10⁸, 10⁸ and 10⁹, and/or 10⁹ and 10¹⁰ cells are delivered. In certain situations, it may be that delivery of cell numbers ranging into the billions may have undesirable effects. Generally, fewer than about 10¹⁰ cells will be delivered.

The concentration of cells delivered will vary depending upon factors such as the total number of cells and the number of delivery sites. According to certain embodiments of the invention cells are delivered at a concentration of approximately 8×10⁷ cells/ml. Of course lower concentrations may be used. Generally, it is preferred to deliver cells at a concentration lower than 16×10⁷ cells/mi. The transplantable compositions may contain skeletal myoblasts and, optionally, fibroblasts in percentages as described above. According to cerrtain embodiments of the invention the compositions are essenetially free of endothelial cells or contain less than approximately 5%, less than approximately 1%, or less than approximately 0.5% endothelial cells.

To administer the compositions, the muscle cells of the invention can be inserted into a delivery device which facilitates introduction by, injection or implantation, of the cardiomyocytes into the subject. Such delivery devices include tubes, e.g., syringes or catheters, for injecting cells and fluids into the body of a recipient subject. In a preferred embodiment, the tubes additionally have a needle through which the cells of the invention can be introduced into the subject at a desired location. The muscle cells of the invention can be inserted into such a delivery device, e.g., a syringe, in different forms. The needle gauge used in transplantation of the cells can be, e.g., 25 to 30.

Cells may be delivered to multiple sites within the heart, e.g., multiple injections can be used. The number of injections may vary depending upon the number of cells delivered and the size of the area to be treated. Cells may be delivered continuously, e.g., along a needle track as the needle is withdrawn or may be delivered in a number of discrete boluses. According to certain embodiments of the invention cells are delivered at a number of locations in the myocardial wall at different depths from the endocardial or epicardial surface. According to certain other embodiments of the invention cells are delivered at multiple locations at approximately the same distance from the endocardial or epicardial surface, e.g., within a particular myocardial layer. According to certain embodiments of the invention some or all of the cells are delivered sub-endocardially or sub-epicardially. According to certain embodiments of the invention some or all of the cells are delivered to the epicardial fat layer.

According to certain embodiments of the invention cellular compositions are delivered in conjunction with, i.e., immediately before, during, or after, another procedure such as placement of a left ventricular assist device (LVAD) or intraaortic balloon pump, coronary artery bypass graft (CAGB), valve replacement, angiography, etc. The area to be treated may be selected visually, e.g., during surgery. Non-invasive techniques such as imaging, including echocardiography and metabolic imaging (e.g., positron emission tomography) may be used to select an appropriate area for treatment.

Cells can be suspended in a solution or embedded in a support matrix when contained in an appropriate delivery device. As used herein, the term “solution” includes a pharmaceutically acceptable carrier or diluent in which the cells of the invention are suspended such that they remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating muscle cells as described herein in a pharmaceutically acceptable carrier or diluent and, as required, other ingredients enumerated above, followed by filtered sterilization.

According to certain embodiments of the invention the composition may contain compounds such as pharmaceuticals (e.g., antibiotic or agents that act on the heart), factors such as growth factors that may stimulate myoblast survival, proliferation, or differentiation, factors that may promote angiogenesis, etc.

In one embodiment, delivery of the cells directly to the damaged area of the heart can be accomplished with a catheter that can reach the ischemic area of the heart and enter the myocardial tissue. For example, a catheter can be introduced percutaneously and routed through the vascular system or by catheters that reach the heart through surgical incisions such as a limited thoracotomy involving an incision between the ribs.

In a preferred embodiment, a type of catheter that is normally not used to deliver cells is used to deliver the muscle cells of the invention (e.g., catheters which are not known in the art to be appropriate for delivery of cells, but which are used to deliver drugs, biologicals, proteins, or genes). Surprisingly, these catheters provide an excellent mechanism by which cells can be delivered to damaged cardiac tissue, even though the damaged cardiac wall can be quite thin. For example, one type of catheter can be introduced into the femoral artery and threaded into the left ventricle where it is used to deliver cells into the heart from the endocardial surface via a needle that is extruded from the end of the catheter. This type of catheter can be localized to the desired area by fluoroscopy (MicroHeart) or by a sensor (Boston Scientific) that aids in targeting cells to the ischemic zone of the myocardium (BioSense). A second type of catheter is introduced via the cardiac venous system (see, e.g., catheters available from Transvascular, Inc. and described at the Web site having URL www.transvascular.com). Cells may be injected into the myocardium from the epicardial side through a needle that is extruded from a housing at the end of the catheter upon reaching the ischemic zone. A multineedle catheter may be introduced via a minithoracotomy and the desired depth, pattern and volume can be set to deliver the cells. These catheters can also be used in conjunction with a laser that is used to create openings in the endocardium that allow better access of the cells and stimulate the growth of new blood vessels in the channels formed by the laser.

Support matrices in which the muscle cells can be incorporated or embedded include matrices which are recipient-compatible and which degrade into products which are not harmful to the recipient. Natural and/or synthetic biodegradable matrices are examples of such matrices. Natural biodegradable matrices include, for example, collagen matrices. Synthetic biodegradable matrices include synthetic polymers such as polyanhydrides, polyorthoesters, and polylactic acid. These matrices provide support and protection for the cardiomyocytes in vivo.

The muscle cells can be administered to a subject by any appropriate route which results in delivery of the cells to a desired location in the subject where they engraft. It is preferred that at least about 5%, preferably at least about 10%, more preferably at least about 20%, yet more preferably at least about 30%, still more preferably at least about 40%, and most preferably at least about 50% or more of the cells remain viable after administration into a subject. The period of viability of the cells after administration to a subject can be as short as a few hours, e.g., twenty-four hours, to a few days, to as long as a few weeks to months, or years.

Once delivered, the ability of the cells and compositions of the invention to enhance cardiac function in a subject can be measured by a variety of means known in the art. For example, the ability of the cells to improve systolic myocardial performance or contractility can be measured. In addition, the cells and compositions of the invention can be tested for their ability to improve the diastolic pressure-strain relationship in the subject. Functional studies such as echocardiography and other imaging studies, performance on stress tests, etc., may be used. Clinical criteria such as dyspnea and chest pain may be assessed. As discussed in more detail elsewhere herein, the ability of the cells and compositions of the invention to survive and engraft within the heart can be assessed using a variety of techniques including histochemistry.

The muscle cells of the invention can further be included in compositions which comprise agents in addition to the muscle cells or muscle cell compositions of the invention. For example, such compositions can include pharmaceutical carriers, antibodies, immunosuppressive agents, or angiogenic factors.

VII. In vivo Applications of the Methods and Compositions of the Invention

As presented in further detail in the Examples, inventors have employed certain of the transplantable compositions and methods described herein in the context of a variety of animal models. In some of these models animals suffer from artificially induced myocardial dysfunction and/or damage. In addition, certain of the transplantable compositions have been delivered to human subjects suffering from cardiac dysfunction or damage, e.g., ischemic damage. As described in Examples 9 and 10, the inventors have provided histologic evidence demonstrating survival and differentiation of human skeletal myoblasts within the human heart as well as evidence demonstrating angiogenesis within regions of damaged myocardial tissue (e.g., scar tissue). To the best of the inventors' knowledge, these results represent the first histological evidence of skeletal myoblast survival and differentiation within the human heart.

Prior to applying the methods and compositions of the invention to human subjects, the inventors undertook extensive investigations in animals. The inventors tested their protocols and approaches in a number of animal models. Animal models are routinely used for predicting effective therapies. Nonetheless, in some instances, results in human are required to establish efficacy. For instance, in some cases, the artificial cardiac injury induced in animal models may not adequately mimic features of ischemic damage, or other naturally-occurring injury, in humans. In particular, the method of creating the injury in the animal frequently involves an invasive process and is often abrupt whereas in human subjects certain types of myocardial damage may be chronic and/or may include both chronic and acute components. Similarly, the time interval between an event causing myocardial damage in animal models and the time at which a cell transplant is performed is frequently shorter than the time interval between myocardial damage and cell transplant in a human subject. Also, myocardial damage in human subjects frequently arises from the presence of a clot within the arterial supply and thus mediators and factors associated with clot formation and resolution are present in the vicinity of the injured myocardium. In addition, interspecies differences between skeletal myoblasts, myotubes, myofibers, fibroblasts, and differentiation processes may mean that cell preparation techniques appropriate for animal cells may not be appropriate for human cells.

The present invention includes the first studies of skeletal myoblast transplantation in human subjects. Experience with certain functional assessments of human subjects allows monitoring for adverse events, such as arrhythmias can be performed. In addition, it is possible to follow a patient's subjective response to therapy. Symptoms such as dyspnea and chest pain and indices of overall well-being can be assessed. For all of the foregoing reasons and many others, the inventive demonstration of safety and efficacy in human subjects provides valuable information for skeletal myoblast transplantation therapies for use in human subjects.

As described in further detail in the Examples, certain transplantable compositions of the invention have been prepared and delivered to human subjects according to the methods described herein. Four of the subjects received the compositions by injection in conjunction with placement of a left ventricular assist device as a bridge to heart transplant. For three subjects the heart was removed at the time of heart transplant and examined for evidence of cellular survival and differentiation. The fourth LVAD recipient awaits transplant. In addition, nine subjects received the compositions by injection in conjunction with CABG procedures. As of Mar. 21, 2002, these subjects are all alive and making satisfactory progress. Tables 6 and 7 present a summary of data relating to each subject including age, transplant date, cell number transplanted (dose), % myoblasts in transplanted composition, whether cells were cryopreserved, number of grafts, date of myocardial infarct (if known), number of injections, and number of adverse events attributable to transplantation. It is envisioned that the transplantable compositions and methods of the invention will be useful in these and a wide variety of other clinical settings ranging from acute myocardial infarction to long-standing cardiac dysfunction due to any cause.

TABLE 6
LVAD Patient Summary

| | | | | | | | | AE | |
|---|---|---|---|---|---|---|---|---|---|
| | | | | | Percent | | | Related | |
| ID | Center | Age | Date | Dose | Myoblasts | Fresh/Cryo | Injections | to Cells | Histology |
| JW-01 | Temple | 43 | Aug. 15, 2000 | 2.2 × 10⁶ | 75% | Fresh | 3 | 0 | (−) |
| FCS-02 | Michigan | 60 | May 4, 2001 | 300 × 10⁶ | 97% | Cryo | 30 | 0 | (+) |
| JDR-03 | Michigan | 62 | Aug. 24, 2001 | 300 × 10⁶ | 62% | Cryo | 17 | 0 | (+) |
| EAG-04 | Michigan | 49 | Jan. 11, 2002 | 300 × 10⁶ | 43% | Cryo | 7 | | ND |

TABLE 7
CABG Patient Summary

| | | | Date of | | % | | # of | Date of | | AE related |
|---|---|---|---|---|---|---|---|---|---|---|
| ID | Center | Age | Transplant | Dose | Myoblast | Fresh/Cryo | Grafts | M.I. | Injections | to cells |
| EJC-01 | UCLA | 61 | May 11, 2001 | 10 × 10⁶ | 92% | Fresh | 3 | 3 MI | 3 | 0 |
| | | | | | | | | March 1978, | | |
| | | | | | | | | '86, April 2001 | | |
| BB-02 | CCF | 52 | Jun. 8, 2001 | 10 × 10⁶ | 64% | Fresh | 2 | ND | 3 | 0 |
| DHS-03 | CCF | 56 | Jul. 10, 2001 | 10 × 10⁶ | 96% | Fresh | 2 | June 1990 | 3 | 0 |
| JEM-04 | CCF | 33 | Oct. 6, 2001 | 30 × 10⁶ | 70% | Cryo | 2 | July 2001 | 3 | 0 |
| SET-05 | AHI | 63 | Dec. 11, 2001 | 30 × 10⁶ | 67% | Cryo | ? | 1991 | 3 | 0 |
| JMS-06 | AHI | 69 | Jan. 3, 2002 | 30 × 10⁶ | 94% | Cryo | ? | ND | 3 | 0 |
| GFM-07 | AHI | 75 | Feb. 13, 2002 | 100 × 10⁶ | 61% | Cryo | ? | October 2001 | 10 | |
| JHG-08 | OSU | 53 | Mar. 12, 2002 | 100 × 10⁶ | 98% | Cryo | 2 | ND | 10 | 0 |
| GRS-09 | AHI | 54 | Mar. 13, 2002 | 100 × 10⁶ | 87% | Cryo | ? | ND | 10 | 0 |

VIII. Modulation of Immune Response

Prior to introduction into a subject, the muscle cells can be modified to inhibit immunological rejection. The muscle cells can, as described in detail herein, be rendered suitable for introduction into a subject by alteration of at least one immunogenic cell surface antigen (e.g., an MHC class I antigen). To inhibit rejection of transplanted muscle cells and to achieve immunological non-responsiveness in an allogeneic or xenogeneic transplant recipient, the method of the invention can include alteration of immunogenic antigens on the surface of the muscle cells prior to introduction into the subject. This step of altering one or more immunogenic antigens on muscle cells can be performed alone or in combination with administering to the subject an agent which inhibits T cell activity in the subject. Alternatively, inhibition of rejection of a muscle cell graft can be accomplished by administering to the subject an agent which inhibits T cell activity in the subject in the absence of prior alteration of an immunogenic antigen on the surface of the muscle cells. As used herein, an agent which inhibits T cell activity is defined as an agent which results in removal (e.g., sequestration) or destruction of T cells within a subject or inhibits T cell functions within the subject (i.e., T cells may still be present in the subject but are in a non-functional state, such that they are unable to proliferate or elicit or perform effector functions, e.g. cytokine production, cytotoxicity etc.). The term “T cell” encompasses mature peripheral blood T lymphocytes. The agent which inhibits T cell activity may also inhibit the activity or maturation of immature T cells (e.g., thymocytes).

A preferred agent for use in inhibiting T cell activity in a recipient subject is an immunosuppressive drug. The term “immunosuppressive drug or agent” is intended to include pharmaceutical agents which inhibit or interfere with normal immune function. A preferred immunsuppressive drug is cyclosporin A. Other immunosuppressive drugs which can be used include FK506, and RS-61443. In one embodiment, the immunosuppressive drug is administered in conjunction with at least one other therapeutic agent. Additional therapeutic agents which can be administered include steroids (e.g., glucocorticoids such as prednisone, methyl prednisolone and dexamethasone) and chemotherapeutic agents (e.g., azathioprine and cyclosphosphamide). In another embodiment, an immunosuppressive drug is administered in conjunction with both a steroid and a chemotherapeutic agent. Suitable immunosuppressive drugs are commercially available (e.g., cyclosporin A is available from Sandoz, Corp., East Hanover, N.J.).

An immunsuppressive drug is administered in a formulation which is compatible with the route of administration. Suitable routes of administration include intravenous injection (either as a single infusion, multiple infusions or as an intravenous drip over time), intraperitoneal injection, intramuscular injection and oral administration. For intravenous injection, the drug can be dissolved in a physiologically acceptable carrier or diluent (e.g., a buffered saline solution) which is sterile and allows for syringability. Dispersions of drugs can also be prepared in glycerol, liquid polyethylene glycols, and mixtures thereof and in oils. Convenient routes of administration and carriers for immunsuppressive drugs are known in the art. For example, cyclosporin A can be administered intravenously in a saline solution, or orally, intraperitoneally or intramuscularly in olive oil or other suitable carrier or diluent.

An immunosuppressive drug is administered to a recipient subject at a dosage sufficient to achieve the desired therapeutic effect (e.g., inhibition of rejection of transplanted cells). Dosage ranges for immunosuppressive drugs, and other agents which can be coadministered therewith (e.g., steroids and chemotherapeutic agents), are known in the art (See e.g., Kahan, B. D. (1989) New Eng. J. Med. 321(25):1725-1738). A preferred dosage range for immunosuppressive drugs, suitable for treatment of humans, is about 1-30 mg/kg of body weight per day. A preferred dosage range for cyclosporin A is about 1-10 mg/kg of body weight per day, more preferably about 1-5 mg/kg of body weight per day. Dosages can be adjusted to maintain an optimal level of the immunosuppressive drug in the serum of the recipient subject. For example, dosages can be adjusted to maintain a preferred serum level for cyclosporin A in a human subject of about 100-200 ng/ml. It is to be noted that dosage values may vary according to factors such as the disease state, age, sex, and weight of the individual. Dosage regimens may be adjusted over time to provide the optimum therapeutic response according to the individual need and the professional judgment of the person administering or supervising the administration of the compositions, and that the dosage ranges set forth herein are exemplary only and are not intended to limit the scope or practice of the claimed composition.

In one embodiment of the invention, an immunsuppressive drug is administered to a subject transiently for a sufficient time to induce tolerance to the transplanted cells in the subject. Transient administration of an immunosuppressive drug has been found to induce long-term graft-specific tolerance in a graft recipient (See Brunson et al. (1991) Transplantation 52:545; Hutchinson et al. (1981) Transplantation 32:210; Green et al. (1979) Lancet 2:123; Hall et al. (1985) J. Exp. Med. 162:1683). Administration of the drug to the subject can begin prior to transplantation of the cells into the subject. For example, initiation of drug administration can be a few days (e.g., one to three days) before transplantation. Alternatively, drug administration can begin the day of transplantation or a few days (generally not more than three days) after transplantation. Administration of the drug is continued for sufficient time to induce donor cell-specific tolerance in the recipient such that donor cells will continue to be accepted by the recipient when drug administration ceases. For example, the drug can be administered for as short as three days or as long as three months following transplantation. Typically, the drug is administered for at least one week but not more than one month following transplantation. Induction of tolerance to the transplanted cells in a subject is indicated by the continued acceptance of the transplanted cells after administration of the immunosuppressive drug has ceased. Acceptance of transplanted tissue can be determined morphologically (e.g., with skin grafts by examining the transplanted tissue or by biopsy) or by assessment of the functional activity of the graft.

Another type of agent which can be used to inhibit T cell activity in a subject is an antibody, or fragment or derivative thereof, which depletes or sequesters T cells in a recipient. Antibodies which are capable of depleting or sequestering T cells in vivo when administered to a subject are known in the art. Typically, these antibodies bind to an antigen on the surface of a T cell. Polyclonal antisera can be used, for example anti-lymphocyte serum. Alternatively, one or more monoclonal antibodies can be used. Preferred T cell-depleting antibodies include monoclonal antibodies which bind to CD2, CD3, CD4 or CD8 on the surface of T cells. Antibodies which bind to these antigens are known in the art and are commercially available (e.g., from American Type Culture Collection). A preferred monoclonal antibody for binding to CD3 on human T cells is OKT3 (ATCC CRL 8001). The binding of an antibody to surface antigens on a T cell can facilitate sequestration of T cells in a subject and/or destruction of T cells in a subject by endogenous mechanisms. Alternatively, a T cell-depleting antibody which binds to an antigen on a T cell surface can be conjugated to a toxin (e.g., ricin) or other cytotoxic molecule (e.g., a radioactive isotope) to facilitate destruction of T cells upon binding of the antibody to the T cells. See U.S. patent application Ser. No.: 08/220,724, filed Mar. 31, 1994, for further details concerning the generation of antibodies which can be used in the present invention.

Another type of antibody which can be used to inhibit T cell activity in a recipient subject is an antibody which inhibits T cell proliferation. For example, an antibody directed against a T cell growth factor, such as IL-2, or a T cell growth factor receptor, such as the IL-2 receptor, can inhibit proliferation of T cells (See e.g., DeSilva, D. R. et al. (1991) J. Immunol. 147:3261-3267). Accordingly, an IL-2 or an IL-2 receptor antibody can be administered to a recipient to inhibit rejection of a transplanted cell (see e.g. Wood et al. (1992) Neuroscience 49:410). Additionally, both an IL-2 and an IL-2 receptor antibody can be coadministered to inhibit T cell activity or can be administered with another antibody (e.g., which binds to a surface antigen on T cells).

An antibody which depletes, sequesters or inhibits T cells within a recipient can be administered at a dose and for an appropriate time to inhibit rejection of cells upon transplantation. Antibodies are preferably administered intravenously in a pharmaceutically acceptable carrier or diluent (e.g., a sterile saline solution). Antibody administration can begin prior to transplantation (e.g., one to five days prior to transplantation) and can continue on a daily basis after transplantation to achieve the desired effect (e.g., up to fourteen days after transplantation). A preferred dosage range for administration of an antibody to a human subject is about 0.1-0.3 mg/kg of body weight per day. Alternatively, a single high dose of antibody (e.g., a bolus at a dosage of about 10 mg/kg of body weight) can be administered to a human subject on the day of transplantation. The effectiveness of antibody treatment in depleting T cells from the peripheral blood can be determined by comparing T cell counts in blood samples taken from the subject before and after antibody treatment. Dosage regimes may be adjusted over time to provide the optimum therapeutic response according to the individual need and the professional judgment of the person administering or supervising the administration of the compositions. Dosage ranges set forth herein are exemplary only and are not intended to limit the scope or practice of the claimed composition.

The present invention is further illustrated by the following examples which in no way should be construed as being further limiting. The contents of all cited references (including literature references, issued patents, published patent applications, and co-pending patent applications) cited throughout this application are hereby expressly incorporated by reference.

### EXAMPLE 1

### Cellular Therapy for Myocardial Repair: Successful Transplantation of Myoblasts by Intracoronary Injection into the Heart after Acute Myocardial Infarction

Cellular transplantation (CT), a potential strategy for myocardial repair, has not been performed in a large animal model of acute myocardial infarction (AMI). The feasibility of CT with human myoblasis (HM) delivered by intracoronary (IC) injection into infarcted canine myocardium in vivo was investigated.

In in vitro studies: cloned HM isolated from skeletal muscle biopsies were cocultured with fetal cardiomyocytes (FC); 2) in vivo studies: Adult mongrel dogs were subject (via left thoracotomy) to left anterior descending coronary artery (LAD) occlusion for 90 min. followed by sustained reperfusion. At 1 hr or 1 day post AMI, CM (40×10⁶ cells) transfected with the reporter gene LacZ were bolused by injection into the LAD. Cyclosporine and prednisone were given daily. At 1 hr or 7 days post transplant, hearts were harvested and serial sections examined for β-gal histochemistry.

In coculture HM showed integration and synchronous contractility with FC. 2) in dogs LacZ positive cells showed a) perivasular infiltration of HM; b) extensive engraftment of HM bordering the AMI zone from epicardium to endocardium; and c) permeation of HM into the AMI zone where new vasculture is developing. Thus, in dogs HM can be implanted and survive in the periphery of infarcted myocardium; 2) CT to augment damages myocardial cells can be performed by IC injection.

### EXAMPLE 2

### Induction of Cardiomyocyte Phenotype in Skeletal Myoblasts Using Cardiomyocyte-Specific GATA4/6 Transcription Factors

In order to address issues concerning the time and mode of myoblast infusion studies were conducted using dog myoblasts under a Cyclosporin A (CyA) and prednisone immunosuppression regimen starting one day before cell transplantation. Dog myoblasts were isolated from male skeletal muscle (TA) biopsies and transplanted into female dogs. Cells for the short-term studies were labeled with CM-DII before transplantation and were detected by fluorescence microscopy. These allogeneic dog myoblast studies (short-term) were proposed to address the time and mode of cell transplantation. The green fluorescent protein (GFP) recombinant adenoviral vector system can be used to provide a powerful detection method for the implanted myoblasts. This approach is highly efficient in infecting the majority (>90%) of the myoblasts with the GFP reporter gene during short incubation at 37° C.

Construction of E 1-deleted recombinant adenoviral vector carrying GFP cDNA is known in the art. Similar constructs containing both the E1- and the E3-deleted recombinant vector containing GFP and GATA cDNA, respectively were made. Once the adenoviral vector infects the myoblasts it is replication defective and unable to re infect additional cells. The GFP cDNA was subcloned between Not 1 and Xho I sites of the bacterial plasmid vector pAd.RSV4, which uses the RSV long-terminal repeat as a promoter and the SV40 polyadenylation signal and contains Ad sequences 0 to 1 and 9 to 16 map units. The plasmid vector was then cotransfected into 293 cells with pJM 17. Recombinant adenoviral vector was then prepared as a high-titer stock by propagation in 293 cells. Viral titer was determined to be 101 pfu/mL by plaque assay.

Additional adenoviral vectors containing the GFP reporter gene as well as the human cardiomyocyte specific transcription factor GATA4 or GATA6 cDNA can be used to infect myoblasts to help differentiate toward a contractile cardiomyocyte phenotype. This includes the endogenous up regulation of the genes encoding the contractile apparatus and the Ca++ATPase associated with cardiac slow twitch (SERCA).

### EXAMPLE 3

### Antigen Masking: Comparison of PT-85 and W6/32 Binding to Human and Porcine Cells

The affinities of PT-85 and W6/32 for human and porcine cells were measured by FACS analysis in a single experiment to limit variations from looking at multiple previous experiments. The affinities of PT-85 for porcine versus human cells were compared. Also compared were PT-85 to W6/32 for reactivity with human cells.

The half-maximal binding of PT-85 to endothelial cells was at 0.007 ug of antibody (10⁵ cells) and to HeLa cells was at 0.005 ug of antibody. The conclusion is that the affinity for cell surface MHC class I is roughly similar for the porcine and human cell.

The relative affinities for the soluble Class 1 molecules (HO) from porcine vs human cells is not the same. PT-85 precipitates the porcine molecule (from PBLs) with a considerably higher apparent affinity than the human molecule (from JY cells). The lack of correlation between the results for the cell surface and soluble MHC molecules is similar to what was seen in the comparisons of PT-85 and 9-3.

The half maximal binding of W6/32 to HeLa cells was at 0.04 ug of antibody as compared to 0.005 ug for PT-85 (105 cells). The affinity of PT-85 is therefore slightly higher than W6/32 for human cells. Both antibodies reached saturation at concentrations approaching 1 ug, but W6/32 showed slightly higher fluorescence intensity.

Using immunoprecipitation on JY cells, the binding of W6/32 to soluble HLA is far stronger than PT-85: a dark band is obtained with W6/32 (2 ug antibody) whereas the band for the same concentration of PT-85 is barely detectable.

The results indicate PT-85 and W6/32 display similar affinities for cell surface HLA, and that antibody binding to soluble MHC molecules is useful for identification of the antigens but not for the determination of relative affinities. The results are consistent with the two color FACS analysis that showed binding of both W6/32 and PT-85 to the same cells, indicating that both epitopes can be masked simultaneously.

### EXAMPLE 4

### Transplantation and Survival of Muscle Cells in Recipient Hearts

The following transplantations of cells all into male Lewis rats were performed.

(A) Cells isolated from syngeneic skeletal muscles and grown on laminin with EGF for only 3 days (without dexamethasone) were transplanted and observed as follows:

7A1: 1 wk frozen heart (12.5 mg/kg CyA+4 mg/kg prednisone) and

7A2: 1 wk frozen heart (no immunosuppression).

(B) Cells isolated from syngeneic skeletal muscles and grown on collagen with FGF for only 3 days were transplanted and observed as follows:

7B1: 1 wk frozen heart (12.5 mg/kg CyA+4 mg/kg prednisone);

7B2: 1 wk frozen heart (no immunosuppression);

7B3: 1 wk formalin fixed heart(12.5 mg/kg CyA+4 mg/kg prednisone); and

7B4: 1 wk formalin fixed heart (no immunosuppression).

Cells for use in this experiment were permitted to undergo less than 20 population doublings it vitro and were not sorted prior to transplantation. Immunosuppression in the animal started day-1. Animals were transplanted at day 0 by injection of 2×10⁵ cells/site (2 needle track/site). Animals were harvested on day 7. Transplantations were sectioned and analyzed by H&E (+trichrome) and immunostained with anti-myogenin (+anti-CD11). All rat heart sections looked very good for cell survival and anti-myogenin staining. No detectable difference between the groups with or without immunosuppression was observed. Larger areas of survival with 10-fold less transplanted cells relative to experiments using purified cells into syngeneic female rat hearts were noted. The results appear in Table 3.

TABLE 3
Rat Myoblast/Myotube Transplantation Results

| | Cell | Survival | Fixation | H&E | Trichrome | Tagged | Myogenin | MY-32 | MF-20 | CD11 |
|---|---|---|---|---|---|---|---|---|---|---|
| Rat | Injection | Time | Procedure | Staining | Staining | Beads | Staining | Staining | Staining | Staining |
| 7A1 | 1.4 × 10⁵ | 1 week | Freeze | + | N/A/ | − | + | N/A | N/A | − |
| | Myoblasts | | | | | | (Good cell | | | (No immune |
| | | | | | | | survival) | | | cells) |
| 7A2 | 1.4 × 10⁵ | 1 week | Freeze | + | N/A/ | − | + | N/A | N/A | − |
| | Myoblasts | | | | | | (Good cell | | | (No immune |
| | | | | | | | survival) | | | cells) |
| 7B1 | 1.4 × 10⁵ | 1 week | Freeze | + | N/A/ | − | + | N/A | N/A | − |
| | Myoblasts | | | | | | (Good cell | | | (No immune |
| | | | | | | | survival) | | | cells) |
| 7B2 | 1.4 × 10⁵ | 1 week | Freeze | + | N/A/ | − | + | N/A | N/A | − |
| | Myoblasts | | | | | | (Good cell | | | (No immune |
| | | | | | | | survival) | | | cells) |
| 7B3 | 1.4 × 10⁵ | 1 week | Formalin | + | Some blue | − | + | N/A | N/A | N/A |
| | Myoblasts | | | | and pink | | (Good cell | | | |
| | | | | | | | survival) | | | |
| 7B4 | 1.4 × 10⁵ | 1 week | Formalin | + | Some blue | − | + | N/A | N/A | N/A |
| | Myoblasts | | | | and pink | | (Good cell | | | |
| | | | | | | | survival) | | | |

### EXAMPLE 5

### Comparison of Transplantation Results With and Without Sorting of Cells Prior to Transplantation

The following transplantations of cells all into male Lewis rats were performed. In this example, subjects were given experimentally induced myocardial infarctions on day 1. Animals were allowed to recover for one week. Transplantation was performed after the one week resting period.

(A) Cells isolated from syngeneic skeletal muscles and grown on laminin with EGF for only 3 days. 2×10⁵ cells/heart were injected (10⁵/site) and observed as follows:

8A 1: 1 wk survival (Freeze)

8A2: 4 wk survival (Freeze)

8A3: 4 wk survival (Formalin)

8A4: 4 wk survival with immunosuppression (48 hr; Freeze)

8A5: 4 wk survival with immunosuppression (Freeze)

(B) Cells isolated from syngeneic skeletal muscles and grown on laminin with EGF, sorted and expanded. 2×10⁵ cells/heart were injected (10⁵/site) and observed as follows:

8B1: 1 wk survival (Freeze)

8B2: 4 wk survival (Freeze)

8B3: 4 wk survival (Formalin)

8B4: 4 wk survival with immunosuppression (Freeze)

(C) Cells isolated from syngeneic skeletal muscles and grown on laminin with EGF, sorted and expanded. 2×10⁶ cells/heart were injected (10⁶/site; 5-10 fold) and observed as follows:

8C 1: 1 wk survival (Freeze)

8C2: 4 wk survival (Freeze)

8C3: 4 wk survival (Formalin)

8C4: 4 wk survival with immunosuppression (Freeze)

Immunosuppression (12.5 mg/kg CyA+4 mg/kg prednisone) for positive control. Cells were cultured for 3 days (i.e., were unsorted and cultured for a limited time in vitro so that they undergo a limited number of population doublings) or sorted and expanded for 6-10 days (sorted). Crude cells were injected 10⁵ cells/site (2 needle track/heart) (12.5 μl/site). Sorted Cells: A comparison was made between 10⁵ cells/site versus 10⁶ cells/site (40 μl/site), i.e. 12.5 μl/site vs. 40 μl/site. A1, B1, and C1 hearts were harvested between 1 and 2 weeks. Remaining hearts were harvested by 4 weeks. Hearts were sectioned and analyzed by H&E (+trichrome). Cells were immunostained with anti-myogenin (+anti-CD11). Results are shown in Table 4.

TABLE 4
Rat Myoblast/Myotube Results

| | Cell | Survival | Fixation | H&E | Myogenin |
|---|---|---|---|---|---|
| Rat | Injection | Time | Procedure | Staining | Staining |
| 8A1 | 2 × 10⁵ | 1 week | Freeze | + | + |
| | Myoblasts | | | (graft) | Myoblasts |
| 8A2 | 2 × 10⁵ | 4 weeks | Freeze | Small graft | |
| | Myoblasts | | | | |
| 8A3 | 2 × 10⁵ | 4 weeks | Formalin | + | |
| | Myoblasts | | | (graft) | |
| 8A4 | 2 × 10⁵ | 4 weeks | Freeze | + | |
| | Myoblasts | | | (graft) | |
| 8A5 | 2 × 10⁵ | 2 days | Freeze | + | + |
| | Myoblasts | | | (graft) | Myoblasts |
| 8B1 | 2 × 10⁵ | 1 week | Freeze | + | + |
| | Myoblasts | | | (graft) | Myoblasts |
| 8B2 | 2 × 10⁵ | 4 weeks | | | |
| | Myoblasts | | | | |
| 8B3 | 2 × 10⁵ | 4 weeks | | | |
| | Myoblasts | | | | |
| 8B4 | 2 × 10⁵ | 4 weeks | | | |
| | Myoblasts | | | | |
| 8C1 | 2 × 10⁶ | 1 week | Freeze | + | + |
| | Myoblasts | | | (graft) | Myoblasts |
| 8C2 | 2 × 10⁶ | 4 weeks | | | |
| | Myoblasts | | | | |
| 8C3 | 2 × 10⁶ | 4 weeks | | | |
| | Myoblasts | | | | |
| 8C4 | 2 × 10⁶ | 4 weeks | | | |
| | Myoblasts | | | | |

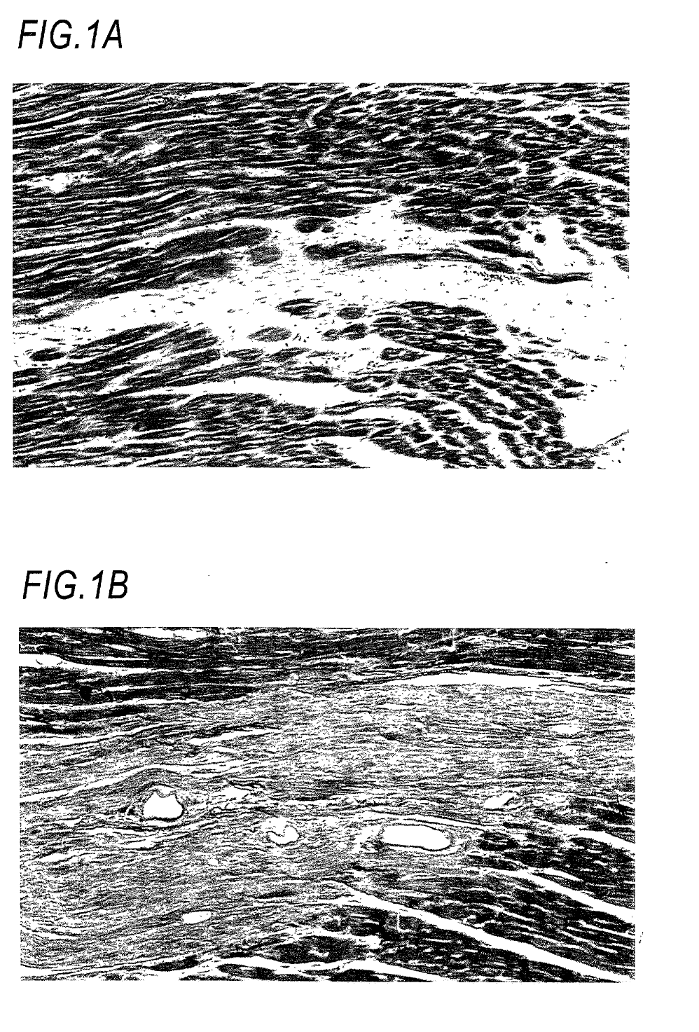

Histology of transplanted grafts indicates that compositions comprising skeletal myoblasts which are permitted to undergo fewer population doublings survive better than such compositions which are sorted to obtain purified cells and permitted to undergo more population doublings. FIG. 1 shows staining of grafts with trichrome. FIG. 1A is a photograph of transplanted cells which were sorted prior to transplantation, while FIG. 1B is a photograph of transplanted cells which were not sorted and were only allowed to undergo several population doublings in vitro prior to transplantation. More grafted cells survive in FIG. 1B.

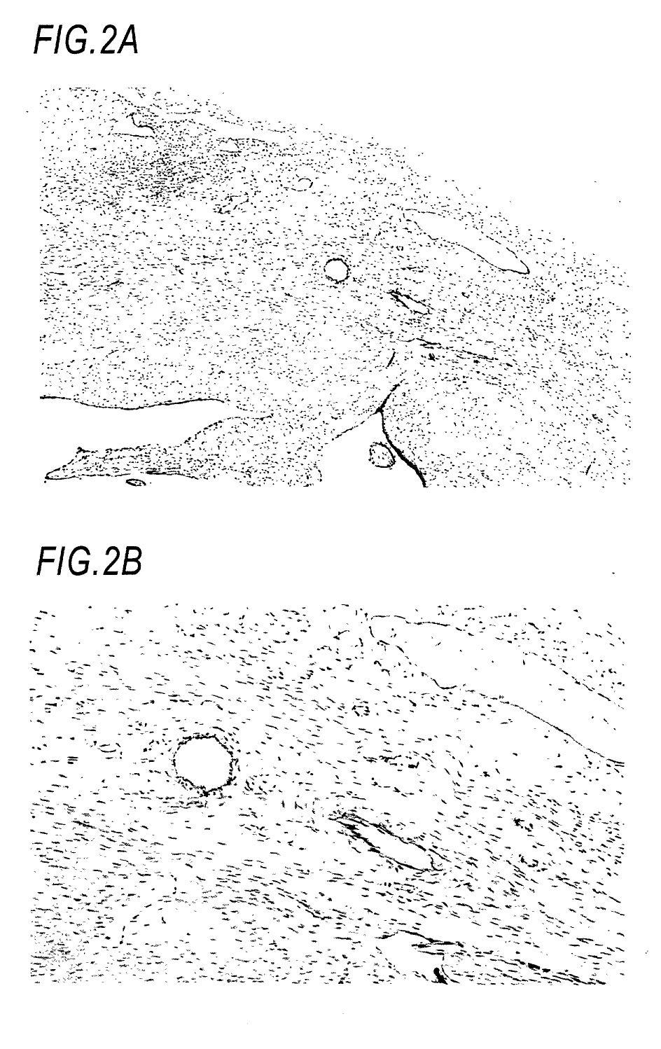

Histological results also indicate that upon the transplantation of compositions comprising skeletal myoblasts into infarcted rat hearts vessel formation (angiogenesis) occurs. FIGS. 2A (lower power) and 2B (higher power) shows staining of such a graft for factor VIII at three weeks post transplantation. Vessels can be seen in the center of the graft.

Exercise max tests were performed on animals which were transplanted with skeletal myoblasts into an infarcted zone in the rat heart. The results of an exemplary test are shown in Table 5. Table 5 compares exercise results for transplanted (myoblast) and control (sham) animals and shows that transplanted animals were able to exercise longer on a treadmill (duration) and go further (distance) than control animals which received a mock transplant.

TABLE 5
Exercise Max Test

| | DURATION (Sec) | DISTANCE (Meters) |
|---|---|---|
| | (mean ± SD) | (mean ± SD) |
| GROUP1 | | |
| (MYOBLAST) | | |
| Baseline | 1144.32 ± 185.87 | 463.54 ± 107.72 |
| (n = 28) | | |
| 3 wk | 1343.31 ± 229.30 | 581.62 ± 140.16 |
| (n + 13) | | |
| GROUP2 | | |
| (SHAM) | | |
| Baseline | 1027.71 ± 106.47 | 395.86 ± 54.73 |
| (n = 7) | | |
| 3 wk | | |
| (n = 7) | 1069.29 ± 145.91 | 443.50 ± 45.18 |

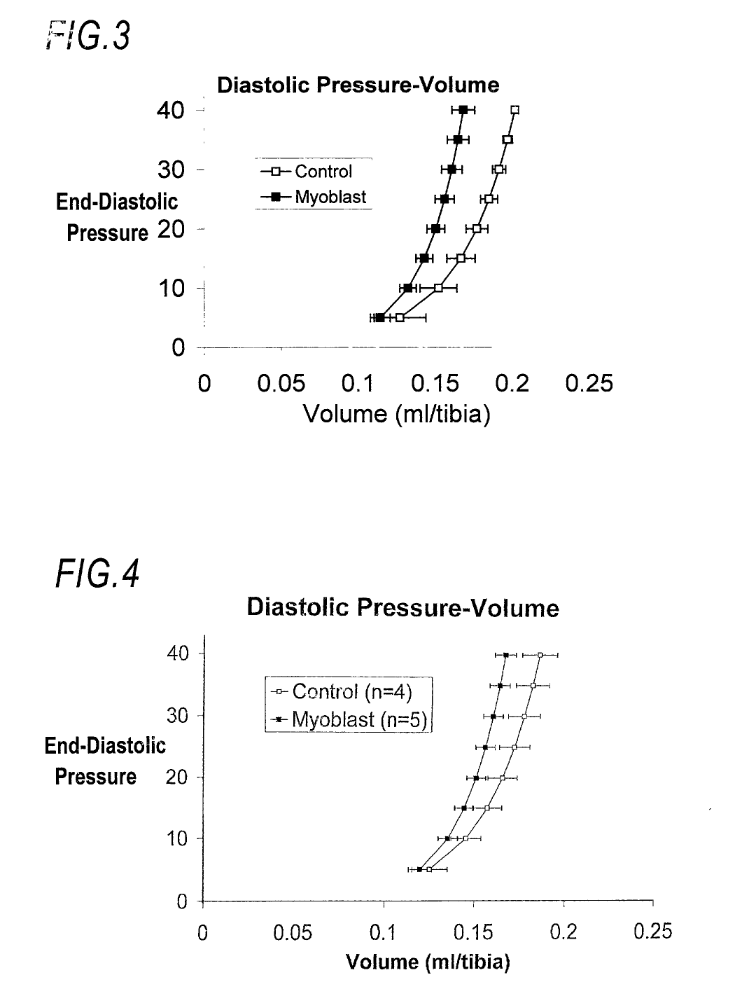

In addition, FIGS. 3 and 4 show that transplanted animals (myoblast) showed improvements in diastolic pressure-volume as compared to nontransplanted control animals. FIGS. 3 and 4 show a reduction in the end-diastolic pressure to volume (corrected for animal size) ratio. These data indicate that the left ventricle of the animals transplanted with myoblasts is being strengthened so that the volume of red blood cells in transplanted hearts is smaller as pressure is increased.

### EXAMPLE 6

### Comparison of Transplantation Results on Ventricular Remodeling and Contractile Function after Myocardial Infarction

The following transplantations of cells into male Lewis rats were performed. In this example, subjects were given experimentally induced myocardial infarctions by coronary ligation on day 1 (see Pfeffer et al. (1979) Circ. Res. 44:503-512; Jain et al. (2000) Cardiovasc. Res. 46:66-72; Eberli et al. (1998) J. Mol. Cell. Cardiol. 30:1443-1447). Animals were allowed to recover for one week. Transplantation was performed after the one week resting period. Myoblasts and fibroblasts isolated from skeletal hind leg muscle of neonatal Lewis rats were isolated and grown on laminin in growth media supplemented with 20% fetal bovine serum for 48 hours. Cells were resuspended in HBSS at 10⁷ cells/mL, and 10⁶ cells/heart were injected (6 to 10 injections) as follows:

(control): non-infarcted control

(MI): myocardial infarction+sham injection

(MI+): myocardial infarction+cell injection

Three groups of animals were studied at three and six weeks following cell therapy.

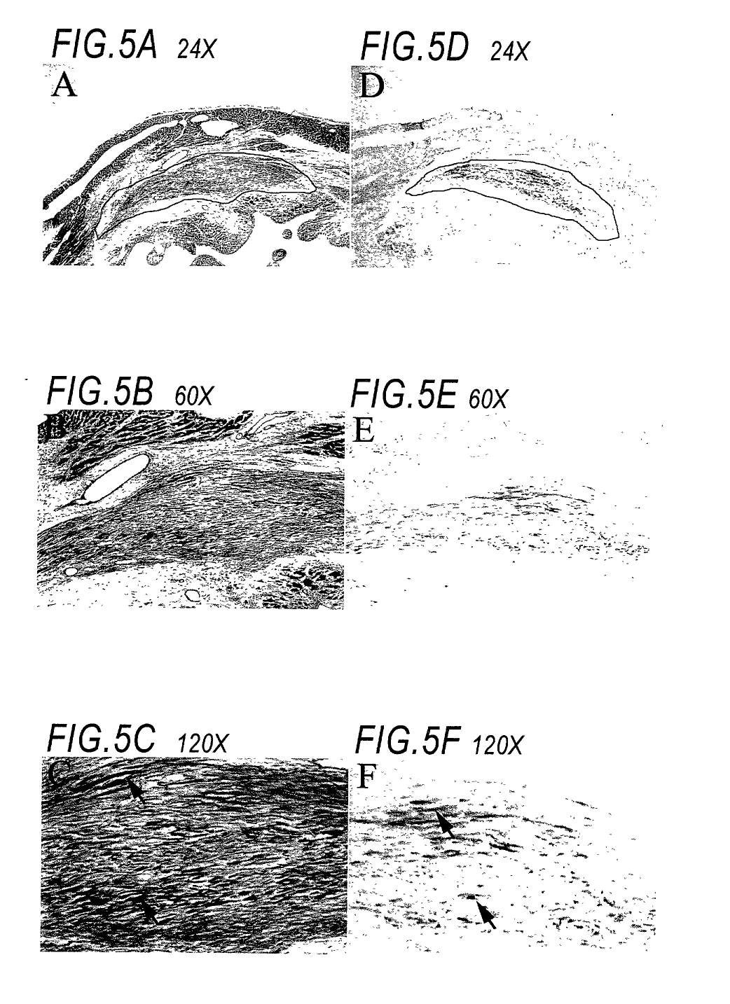

Graft survival was assessed by trichome staining and immunocytochemistry for detection of skeletal myoblasts (anti-myogen stain) and mature myoblasts (anti-skeletal myosin stain). Graft survival was verified at 9 days (FIG. 5) following myoblast implantation. Myogenin positive staining was observed as early as 9 days post-implantation (FIGS. 5D-F), while skeletal myosin heavy chain expression was not observed until three weeks post-implantation. Myoblast survival was confirmed in 6 of 7 and in 9 of 9 animals at three and six weeks post-therapy, respectively. Animals undergoing syngenic cell therapy displayed no evidence for cell rejection, as determined by weight loss, additional mortality or macrophage accumulation in tissue sections.

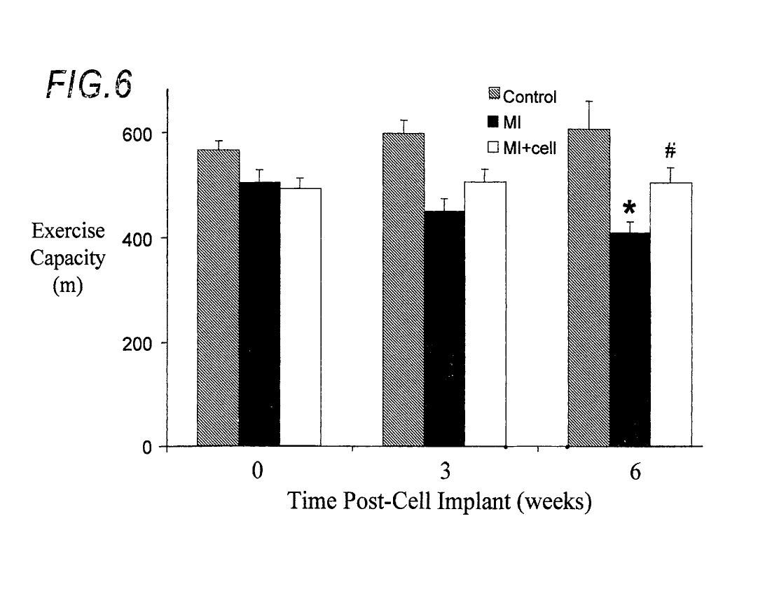

Maximum exercise capacity, a measure of in vivo ventricular function and overall cardiac performance, was determined in all animals prior to cellular implantation (one week post-MI), as well as three and six weeks post-therapy (FIG. 6). MI animals exhibited a gradual decline in exercise performance with time, showing a greater than 30 percent reduction in exercise capacity relative to control animals at six weeks. Cell therapy (MI+) prevented the continued decline of post-MI exercise capacity, suggesting a protection against the progressive deterioration of in vivo cardiac function.

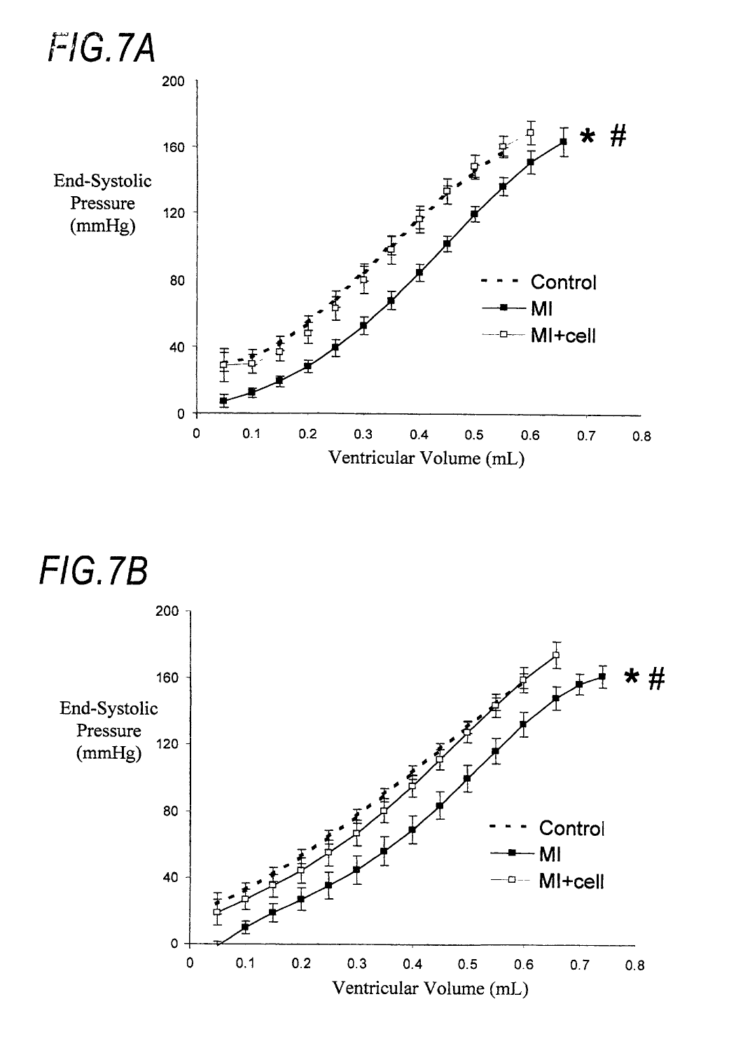

Cardiac contractile function, measured using systolic pressure-volume curves, was assayed by whole heart Langendorff perfusion studies in isolated isovolumically beating hearts (as described in Jain et al. (2000) Cardiovasc. Res. 46:66-72; Eberli et al. (1998) J. Mol. Cell. Cardiol. 30:1443-1447) (FIG. 7). Non-infarcted control hearts exhibited a typical rise in systolic pressure with increasing ventricular volume. Three weeks post-implantation, MI hearts displayed a rightward shift in the systolic pressure-volume curve (FIG. 7A). Cell implantation prevented this shift in MI+hearts, resulting in greater systolic pressure generation at any given preload (ventricular volume). There was, however, no significant difference in the peak systolic pressure generated at maximum ventricular volume (at an end diastolic pressure of 40 mmHG) among groups. The beneficial effects of cell therapy were also observed at six weeks post-therapy (FIG. 7B), suggesting an improvement of ex-vivo global contractile function with myoblast implantation.

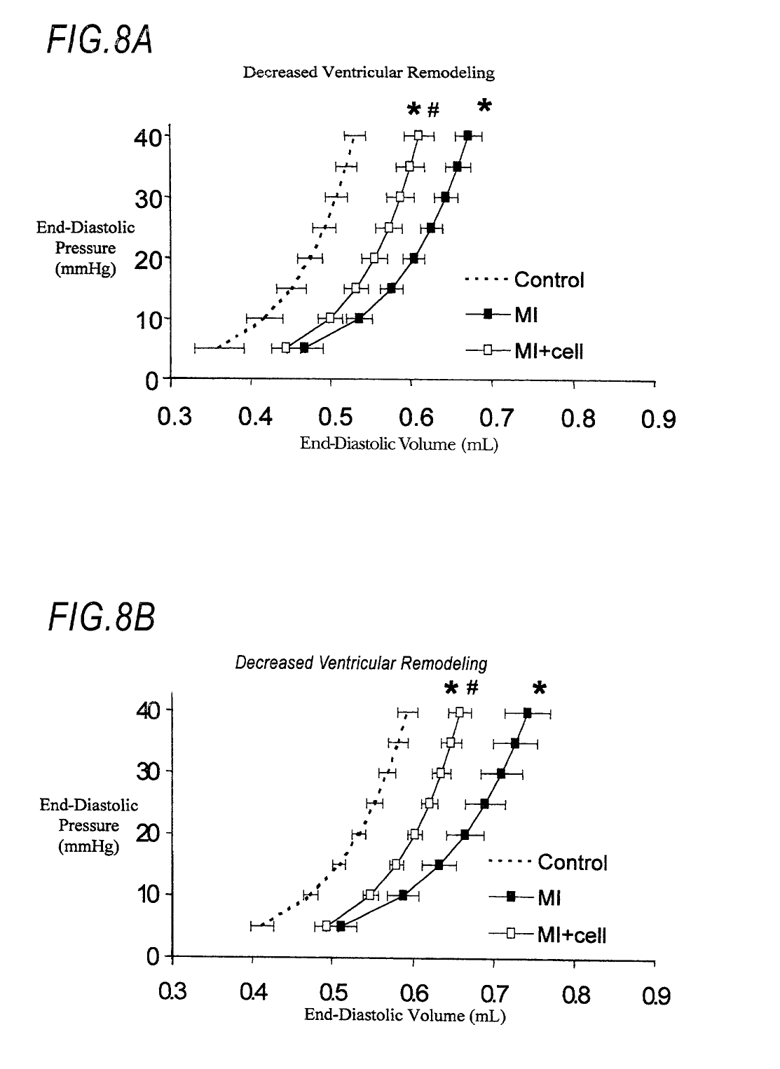

In addition to pump dysfunction, ventricular remodeling characteristically results in progressive global cavity enlargement. Ventricular dilation was assessed with diastolic pressure-volume relationships, established in isolated hearts through monitoring of distending pressures over a range of diastolic volumes (as described in Jain et al.; Eberli et al., supra) (FIG. 8). At all time points, MI hearts exhibited substantially enlarged left ventricles relative to non-infarcted control hearts at any given distending pressure, demonstrated by a rightward repositioning of the pressure-volume curve. Cell therapy, however, caused a significant reduction in ventricular cavity dilation, placing hearts from the MI+group significantly leftward of MI group at both three and six weeks post-implantation, suggesting an attenuation of deleterious post-myocardial infarction ventricular remodeling with cell implantation.

Ventricular remodeling was further investigated through morphometric analysis of tissue sections. At all time points, MI and MI+hearts exhibited enlarged chamber diameters compared to non-infarcted control hearts. Six weeks following cell therapy, hearts from the MI+group had a reduced endocardial cavity diameter relative to MI hearts, suggesting an attenuation of ventricular dilation, similar as observed with diastolic pressure-volume curves in FIG. 8B. In addition, MI hearts exhibited a decrease in infarct wall thickness at both three and six weeks post-therapy, suggesting characteristic post-myocardial infarction scar thinning and infarct expansion. MI+hearts, however, had no significant reduction in infarct wall thickness relative to non-infarcted control hearts. Septal wall thickness was comparable among all groups at both three and six weeks post-therapy. These data indicate that myoblast implantation following MI improves both in vivo and ex vivo indices of global ventricular dysfunction and deleterious remodeling and suggests cellular implantation may be beneficial post-MI.

### EXAMPLE 7

### Autologous Myoblast and Fibroblast Transplantation for the Treatment of End-Stage Heart Disease

Autologous myoblasts and fibroblasts derived from skeletal muscle are transplanted into the myocardium of subjects in end stage heart failure. The human subjects in the study are candidates for heart transplant surgery and are scheduled for placement of a left ventricular assist device as a bridge to orthotopic transplantation.

Prior to transplant, myoblasts and fibroblasts are expanded in vitro from satellite cells obtained from a biopsy of the subject's skeletal muscle. The composition of the cells is preferably 40-60% myoblasts. The cells, at a concentration of 8×10⁷ cells per ml, are injected into the peri-infarct zone of the left ventricle. Injections of up to 100 μl are made into up to 35 sites, with a maximum of 300×10⁶ cells injected.

The safety of myoblast and fibroblast transplantation is assayed based upon unexpected adverse effects, such as abnormal cardiac function. Preliminary information on the autologous graft survival and the potential for improvement of cardiac function that might be associated with the autologous myoblast and fibroblast transplantation is obtained.

### EXAMPLE 8

### Autologous Myoblast and Fibroblast Transplantation for the Treatment of Infarcted Myocardium

Autologous myoblasts and fibroblasts derived from skeletal muscle are transplanted into and around the ischemic or scarred areas of the myocardium, post myocardial infarction. The human subjects in the study have a myocardial infarction and have additional cardiac disease consisting of left ventricular dysfunction that places the subject in the high risk group of candidates for coronary artery bypass graft.

Prior to transplant, myoblasts and fibroblasts are expanded in vitro from satellite cells obtained from a biopsy of the subject's skeletal muscle. The composition of the cells is preferably 40-60% myoblasts. The cells, at a concentration of 8×10⁷ cells per ml, are injected into and around the infarct site in a region of the wall of the left ventricle that has adequate perfusion. Injections of up to 100 μl are made into up to 30 sites.

The safety of myoblast and fibroblast transplantation is assayed based upon adverse events due the transplanted cells and the transplantation procedure. Echocardiography and magnetic resonance imaging are used to evaluate regional wall motion, an assay to detect improvement of cardiac function.

### EXAMPLE 9

### Survival of Autologous Myoblasts Transplanted into Infarcted Human Myocardium

Materials and Methods

This example describes a study in which autologous skeletal myoblasts were isolated from a human subject, processed and expanded in tissue culture, and then delivered to the patient's heart while the patient was undergoing implantation of a left ventricular assist device (LVAD) while awaiting heart transplantation. The Clinical Phase I study was approved by the Institutional Review Board for Human Studies (University of Michigan) and was conducted in accordance with federal guidelines under an approved IND and informed consent process. At the time of heart transplantation the patient's heart was retrieved, and analyzed. These studies provided, for the first time, histological and pathological evidence of survival and engraftment of skeletal myoblasts into a human heart.

Study Subject and Protocol: The patient (subject FCS-02 in Table 6) was a 60 year-old male with a history of ischemic cardiomyopathy (left ventricular ejection fraction 15%), prior coronary artery bypass grafting in 1986, and severe native and graft coronary artery disease not amenable to revascularization. The patient was evaluated and approved for heart transplantation and underwent study recruitment and muscle biopsy. The muscle biopsy was taken from the right quadriceps muscle under sterile conditions using local anesthetics. The muscle specimen was immediately placed in transport medium and sent to the GMP isolation facility.

Four weeks after transplant listing, the patient developed refractory hypotension and nonsustained ventricular tachycardia. He was evaluated and underwent HeartMate® LVAD (Thoratec, Inc.) implantation as a bridge to heart transplantation. At the time of LVAD implantation, multiple injections of autologous skeletal myoblasts were made into the anterior wall of the left ventricle using a 0.5 inch long 26 gauge needle. Injection location was selected based upon echocardiography prior to surgery, and direct visualization during the open heart surgery. Fifteen 100 μl injections were delivered at a constant slow rate of delivery. An additional fifteen 100 μl injections were delivered approximately 1 cm apart with a one-inch long 25-gauge needle. All of the injections were made into a designated area of approximately 3×3 cm² demarcated with surgical clips. The LVAD implant procedure was completed in the usual fashion. The patient recovered uneventfully and was discharged to home on postoperative day 18 with LVAD support. Ninety-one days following LVAD implantation, the patient underwent LVAD explantation and heart transplantation. At the time of operation, the portion of the left ventricle demarcated by the surgical clips was excised, and stored in formalin solution prior to histological analysis. The patient's postoperative course was remarkable for renal insufficiency. The patient improved and was discharged to home in satisfactory condition on postoperative day 30. The patient is alive and well 10 months following transplantation.

Preparation of the Autologous Skeletal Myoblasts: The starting 1.53 grams of skeletal muscle obtained at biopsy was stripped of connective tissue, minced into a slurry in digestion medium, and then subjected to several cycles of enzymatic digestion at 37° C. with 1×trypsin/EDTA (0.5 mg/ml trypsin, 0.53 mM EDTA; GibcoBRL) and collagenase-hepatocyte qualified (0.5 mg/ml; GibcoBRL) to release satellite cells. Skeletal myoblast cultures were expanded according to a modified Ham's method²⁷. Satellite cells were plated and grown in myoblast basal growth medium (SkBM; Clonetics) containing 15-20% fetal bovine serum (Hyclone), recombinant human epidermal growth factor (rhEGF: 10 ng/mL), and dexamethasone (3 μg/mL). A portion of the cells were grown for 10 doublings, and then cryopreserved. After thaw, these cells were combined with a second group of cells which had been grown for 14 doublings to achieve the final yield of 308 million cells. To avoid any possibility of myotube formation during the culture process, cell densities were maintained throughout the process so that <75% of the culture surface was occupied by cells.

Myoblast purity was measured by reactivity with anti-NCAM monoclonal Ab (5.1H11, supplied by Dr. Robert Brown) using Fluorescence Activated Cell Sorting (FACS). This antibody selectively stains human myoblasts and not fibroblasts 28 The ability of myoblasts to fuse into multinucleated myotubes in vitro was also confirmed by seeding 2×10⁵ cells per 24 well tissue culture plate in growth medium. The following day the culture medium was switched to fusion medium (Dulbecco's minimal essential medium+0.1% bovine serum albumin and 50 ng/ml IGF), and cultures were observed three days later to assess fusion. Prior to transplantation, in excess of 300 million cells were washed and suspended in transplantation medium at approximately 100 million cells per cc and loaded into five 1 cc tuberculin syringes. The cells were kept at 4° C. during transport. Sterility tests were conducted on the final product as well as throughout the digestion and expansion procedures.

Histological Analysis and Immunohistochemical Techniques: Excised myocardium was fixed in formalin, cut into small blocks, and paraffin embedded. Six micron thick sections were cut, mounted, and stained with trichrome. For detection of myosin heavy chain, deparaffinized sections were incubated with alkaline phosphatase-conjugated MY-32 mAb (Sigma), a skeletal muscle reactive anti-myosin heavy chain antibody that does not stain cardiac muscle²⁹. Sections were developed with BCIP-NBT (Zymed Lab Inc) and counter stained with nuclear red. For detection of vascular endothelial cells with anti-CD-31 mAb (Clone JC/70A, DAKO Corp.) and T cells with polyclonal rabbit anti-human CD3 antibody (DAKO Corp.), deparaffinized sections were primary antibody according to manufacturer's recommendations. Incubation with secondary antibodies were performed according to instructions for Vectastain Mouse Elite or Vectastain Goat Elite Horse Radish Peroxidase conjugates (Vector Laboratories). Sections were developed with diaminobenzidine (DAB Substrate Kit; Vector Laboratories) and counter stained with hematoxylin.

For vascular endothelium quantitation, a total of six independent locations within the implanted region were immunostained with antiCD-31 mAb. Counts were performed in the region of the grafted cells and in the non-transplanted scar region immediately adjacent to the graft. Each field was photographed using an Olympus microscope with a 20×objective and a Kodak digital camera. The image was then acquired in Photoshop 5.0 and the entire field counted for individual vascular elements. Counts were analyzed and statistical analysis performed by analysis of variance (ANOVA). Statistical significance was defined at p<0.05.

Results

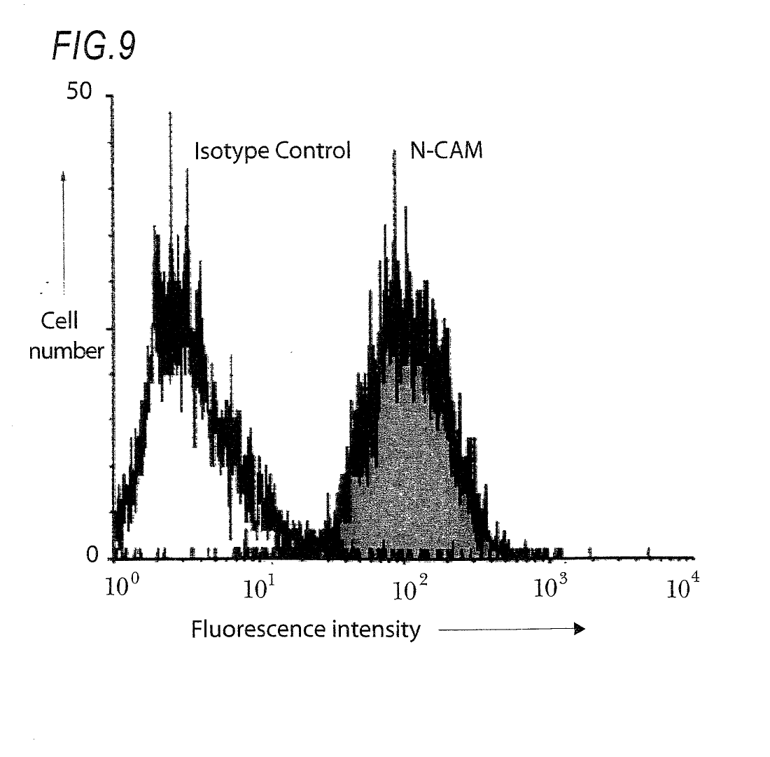

Skeletal myoblasts were expanded in culture with an average doubling time of 29 hours. Prior to transplantation, the population of 308×10⁶ cells contained only single cells and no fused myoblasts. The cell preparation was composed of 96.5% myoblasts based on skeletal muscle-specific anti-NCAM mAb staining and FACS analysis (FIG. 9), with the remainder of the cells being composed of fibroblasts. The final myoblast preparation was characterized further by demonstrating the capacity to fuse and form multinucleated myotubes.

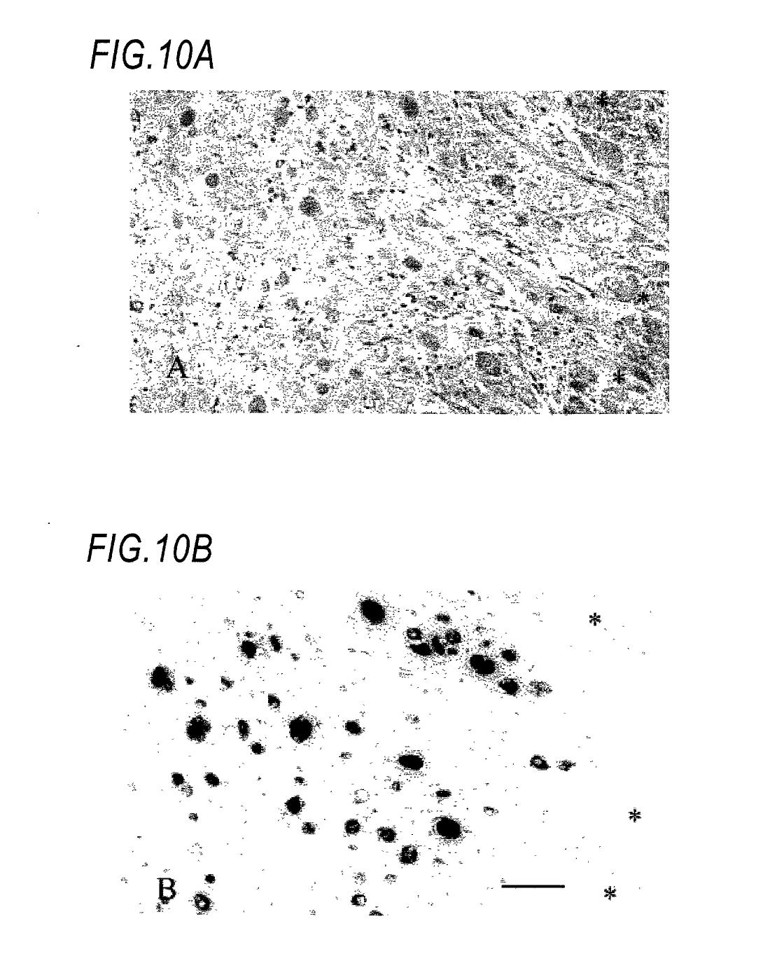

Approximately 300×10⁶ cells were transplanted using multiple injections into the left ventricular wall of the patient. At the time of orthotopic heart transplantation, approximately 3 months after cells were implanted, the explanted heart was fixed and sectioned. Surviving autologous skeletal muscle cells were identified in heavily scarred tissue of the heart by trichrome staining (FIG. 10A). Myofiber structures were identified within the transplant region by the red trichrome stain characteristic of cardiac and skeletal muscle as opposed to the blue stain associated with fibroblasts and collagen of the scar (FIG. 10A). Myofibers continued throughout several blocks of tissue spanning an area of approximately 1.2 cm in length and 2 cm in width. The red stained myofiber tissue was confirmed to be of skeletal origin by staining for skeletal muscle-specific myosin heavy chain (FIG. 10B). Only the transplanted skeletal muscle fibers stain for muscle-specific myosin heavy chain and not the host cardiac muscle fibers. Additionally, most of the transplant-associated myofibers were aligned in parallel with the host myocardial fibers. No difference in morphology or survival of transplanted cells was noted between implants within scarred myocardium or adjacent to healthy myocardium (FIGS. 10A and B).

H&E staining was performed in addition to trichrome staining to better assess the presence of inflammatory cells associated with the grafts of autologous myoblasts. There was no evidence of immune reaction or lymphocyte infiltration associated with either grafted and non-grafted areas. This conclusion was confirmed with T-cell specific anti-CD3 polyclonal antibody immunohistochemical staining (data not shown). However, there were a number of examples of multinucleated giant cells detected in and around the grafts, but not in non-grafted myocardium. The giant cells were seen in association with refractile material likely introduced during injection of cells. There was no evidence of giant cells associated with the transplanted myofibers themselves.

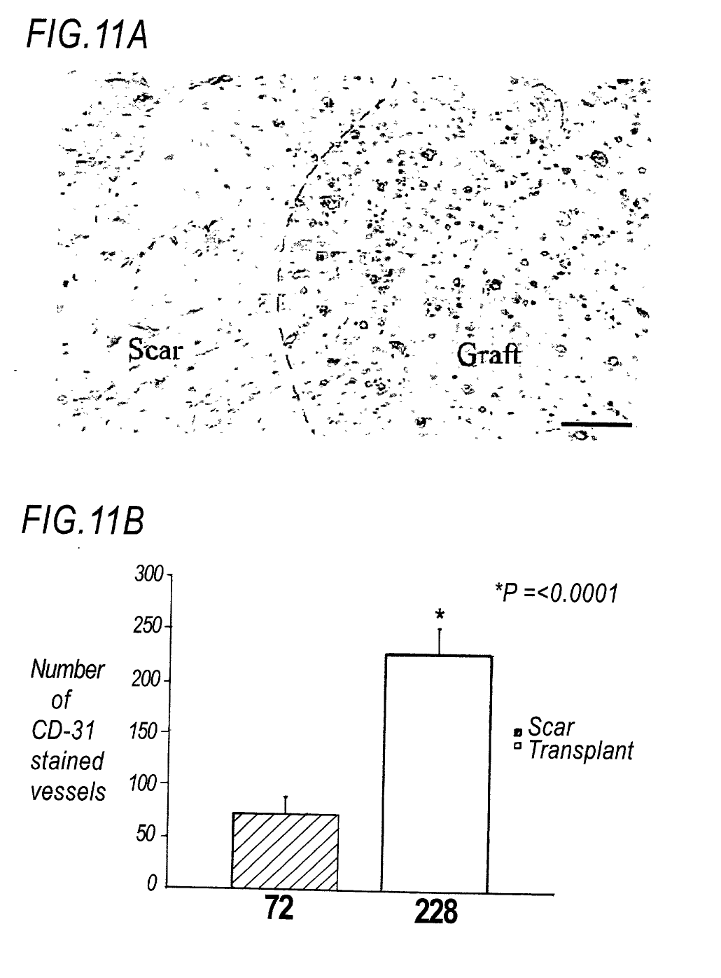

Immunohistochemical staining was also performed to assess the presence of vascular endothelium in grafted and non-grafted myocardium using an anti-CD-31 mAb (FIG. 11). Quantitative measurement from six independent graft areas showed significantly more CD-31 stained vessels at the sites of surviving grafts (FIGS. 11A and B) as compared to the number of vessels in the corresponding non-grafted scar tissue (228±24 cells per field in grafted area vs. 72±17 cells per field in non-grafted area, respectively; p<0.0001) (FIG. 11B).

In summary, the results described above indicate extensive skeletal myoblast survival and differentiation into skeletal myofibers within both scarred and healthy myocardium following delivery to a human heart. In addition, the results indicate that significant angiogenesis occurred within the regions of cell survival. These results confirm the feasibility of skeletal myoblast therapy for human cardiac damage or dysfunction.

References for Example 9

1. Jackson K A, Majka S M, Wang H, et al. Regeneration of ischemic cardiac muscle and vascular endothelium by adult stem cells. [see comments]. Journal of Clinical Investigation 2001; 107:1395-402.

2. Kamihata H, Matsubara H, Nishiue T, et al. Implantation of bone marrow mononuclear cells into ischemic myocardium enhances collateral perfusion and regional function via side supply of angioblasts, angiogenic ligands, and cytokines. Circulation 2001; 104:1046-52.

3. Kocher A A, Schuster M D, Szabolcs M J, et al. Neovascularization of ischemic myocardium by human bone-marrow-derived angioblasts prevents cardiomyocyte apoptosis, reduces remodeling and improves cardiac function. [see comments]. Nature Medicine 2001; 7:430-6.

4. Menasche P, Hagege A A, Scorsin M, et al. Myoblast transplantation for heart failure. Lancet 2001; 357:279-80.

5. Orlic D, Kajstura J, Chimenti S, et al. Bone marrow cells regenerate infarcted myocardium. [see comments]. Nature 2001; 410:701-5.

6. Orlic D, Kajstura J, Chimenti S, et al. Mobilized bone marrow cells repair the infarcted heart, improving function and survival. Proceedings of the National Academy of Sciences of the United States of America 2001; 98:10344-9.

7. Taylor D A, Atkins B Z, Hungspreugs P, et al. Regenerating functional myocardium: improved performance after skeletal myoblast transplantation [published erratum appears in Nat Med October 1998; 4(10):1200]. Nat Med 1998; 4:929-33.

8. Kessler P D, Byrne B J. Myoblast cell grafting into heart muscle: cellular biology and potential applications. Annu Rev Physiol 1999; 61:219-42.

9. Klug M G, Soonpaa M H, Koh G Y, Field L J. Genetically selected cardiomyocytes from differentiating embronic stem cells form stable intracardiac grafts. Journal of Clinical Investigation 1996; 98:216-24.

10. Yoo K J, Li R K, Weisel R D, et al. Heart cell transplantation improves heart function in dilated cardiomyopathic hamsters. Circulation 2000; 102:III204-9.

11. Koh G Y, Soonpaa M H, Klug M G, et al. Stable fetal cardiomyocyte grafts in the hearts of dystrophic mice and dogs. Journal of Clinical Investigation 1995; 96:2034-42.

12. Li R K, Jia Z Q, Weisel R D, et al. Cardiomyocyte transplantation improves heart function. Annals of Thoracic Surgery 1996; 62:654-60; discussion 660-1.

13. Scorsin M, Hagege A A, Marotte F, et al. Does transplantation of cardiomyocytes improve function of infarcted myocardium? Circulation 1997; 96:II-188-93.

14. Scorsin M, Hagege A A, Dolizy I, et al. Can cellular transplantation improve function in doxorubicin-induced heart failure? Circulation 1998; 98:II151-5; discussion II155-6.

15. Soonpaa M H, Koh G Y, Klug M G, Field L J. Formation of nascent intercalated disks between grafted fetal cardiomyocytes and host myocardium [see comments]. Science 1994; 264:98-101.

16. Jain M, DerSimonian H, Brenner D A, et al. Cell therapy attenuates deleterious ventricular remodeling and improves cardiac performance after myocardial infarction. Circulation 2001; 103:1920-7.

17. Hutcheson K A, Atkins B Z, Hueman M T, Hopkins M B, Glower D D, Taylor D A. Comparison of benefits on myocardial performance of cellular cardiomyoplasty with skeletal myoblasts and fibroblasts. Cell Transplant 2000; 9:359-68.

18. Atkins B Z, Hueman M T, Meuchel J, Hutcheson K A, Glower D D, Taylor D A. Cellular cardiomyoplasty improves diastolic properties of injured heart. Journal of Surgical Research 1999; 85:234-42.

19. Murry C E, Wiseman R W, Schwartz S M, Hauschka S D. Skeletal myoblast transplantation for repair of myocardial necrosis. J Clin Invest 1996; 98:2512-23.

20. Reinecke H, MacDonald G H, Hauschka S D, Murry C E. Electromechanical coupling between skeletal and cardiac muscle. Implications for infarct repair. Journal of Cell Biology 2000; 149:731-40.

21. Chiu R C, Zibaitis A, Kao R L. Cellular cardiomyoplasty: myocardial regeneration with satellite cell implantation. [see comments]. Annals of Thoracic Surgery 1995; 60:12-8.

22. Tomita S, Li R K, Weisel R D, et al. Autologous transplantation of bone marrow cells improves damaged heart function. Circulation 1999; 100:11247-56.

23. Pouzet B, Vilquin J T, Hagege A A, et al. Intramyocardial transplantation of autologous myoblasts: can tissue processing be optimized? Circulation 2000; 102:111210-5.

24. Pouzet B, Vilquin J T, Hagege A A, et al. Factors affecting functional outcome after autologous skeletal myoblast transplantation. Annals of Thoracic Surgery 2001; 71:844-50; discussion 850-1.

25. Scorsin M, Hagege A, Vilquin J T, et al. Comparison of the effects of fetal cardiomyocyte and skeletal myoblast transplantation on postinfarction left ventricular function. J Thorac Cardiovasc Surg 2000; 119:1169-75.

26. Menesché P, J. -T. Vilquin, Desnos M, et al. Early results of autologous skeletal myoblast transplantation in patients with severe ischemic heart failure. Circulation 2001; 104:II-598.

27. Ham R G, St Clair J A, Meyer S D. Improved media for rapid clonal growth of normal human skeletal muscle satellite cells. Adv Exp Med Biol 1990; 280:193-9.

28. Webster C, Pavlath G K, Parks D R, Walsh F S, Blau H M. Isolation of human myoblasts with the fluorescence-activated cell sorter. Exp Cell Res 1988; 174:252-65.

29. Havenith M G, Visser R, Schrijvers-van Schendel J M, Bosman F T. Muscle fiber typing in routinely processed skeletal muscle with monoclonal antibodies. Histochemistry 1990; 93:497-9.

### EXAMPLE 10

### Survival of Autologous Myoblasts Transplanted into Infarcted Human Myocardium

Materials and Methods

Study Subject and Protocol: The patient (subject JDR-03 in Table 6) is a 62 year-old male with a diagnosis of ischemic cardiomyopathy and symptoms of heart failure, New York Heart Association class III, with repeated episodes of pulmonary edema that necessitated hospitalization. His past medical history is significant for hypertension, myocardial infarction, and coronary artery bypass surgery in 1992. The patient was evaluated and approved for heart transplantation and underwent study recruitment and muscle biopsy. The muscle biopsy was taken from the right quadriceps muscle under sterile conditions using local anesthetics. The muscle specimen was immediately placed in transport medium and sent to the GMP isolation facility.

Subsequent to removal of the muscle sample, the patient was evaluated and underwent HeartMate® LVAD (Thoratec, Inc.) implantation as a bridge to heart transplantation. At the time of LVAD implantation, multiple injections of autologous skeletal myoblasts were made in a similar fashion to that described in Example 9. The LVAD implant procedure was completed in the usual fashion. The patient recovered uneventfully and was discharged to home with LVAD support. Four months following LVAD implantation, the patient underwent LVAD explantation and heart transplantation. At the time of operation, the portion of the heart demarcated by the surgical clips was excised, and stored in formalin solution prior to histological analysis. The patient improved and was discharged to home in satisfactory condition. The patient is alive and well 7 months following transplantation.

Preparation of the Autologous Skeletal Myoblasts and Histological Analysis and Immunohistochemical Techniques. These were performed similarly to described in Example 9 except that the percent myoblasts was approximately 62% and a total of 17 injections were performed. The concentration of cells was approximately 100 million per cc. Of the 17 injections, 7 injections of 100 μl, 4 injections of 2×100 μl, 4 injections of 4×100 μl, and 2 injections of 4×100 μl were performed, for a total volume injected of 3.5 ml. Quantitative assessments were performed as described in Example 9.

Results

Skeletal myoblasts were expanded in culture as described above. Prior to transplantation, the population of approximately 300×10⁶ cells contained only single cells and no fused myoblasts. The cell preparation was composed of 62% myoblasts based on skeletal muscle-specific anti-NCAM mAb staining and FACS analysis, with the remainder of the cells being composed of fibroblasts. The final myoblast preparation was characterized further by demonstrating the capacity to fuse and form multinucleated myotubes.

Approximately 300×10⁶ cells were transplanted using multiple injections. At the time of orthotopic heart transplantation, approximately four months after cells were implanted, the explanted heart was fixed and sectioned. The tissue was analyzed as described in Example 9.

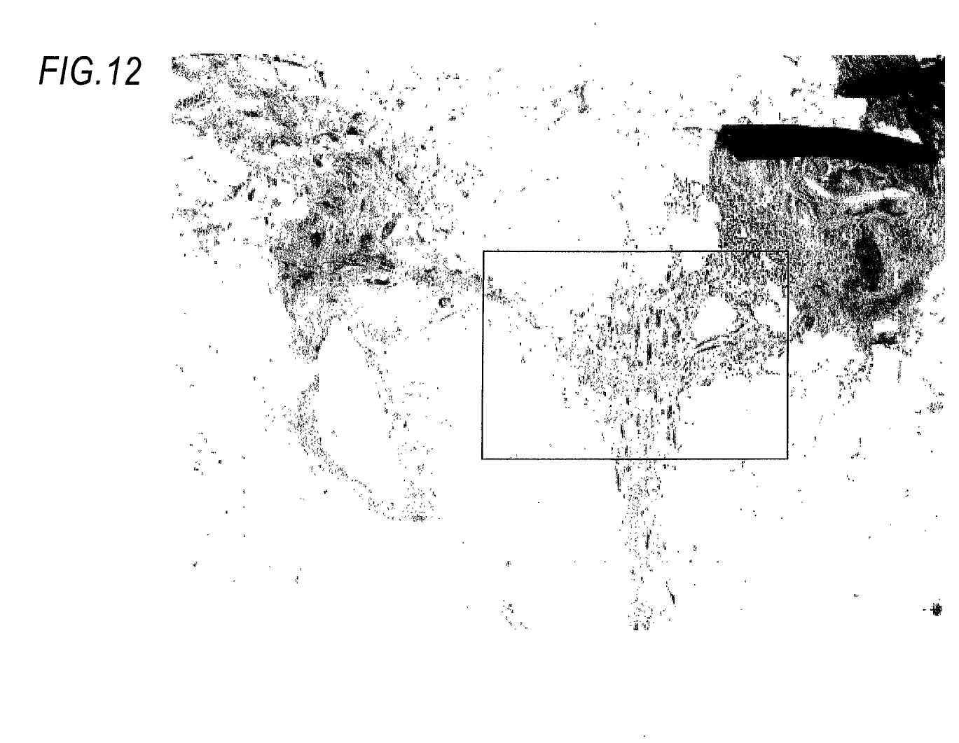

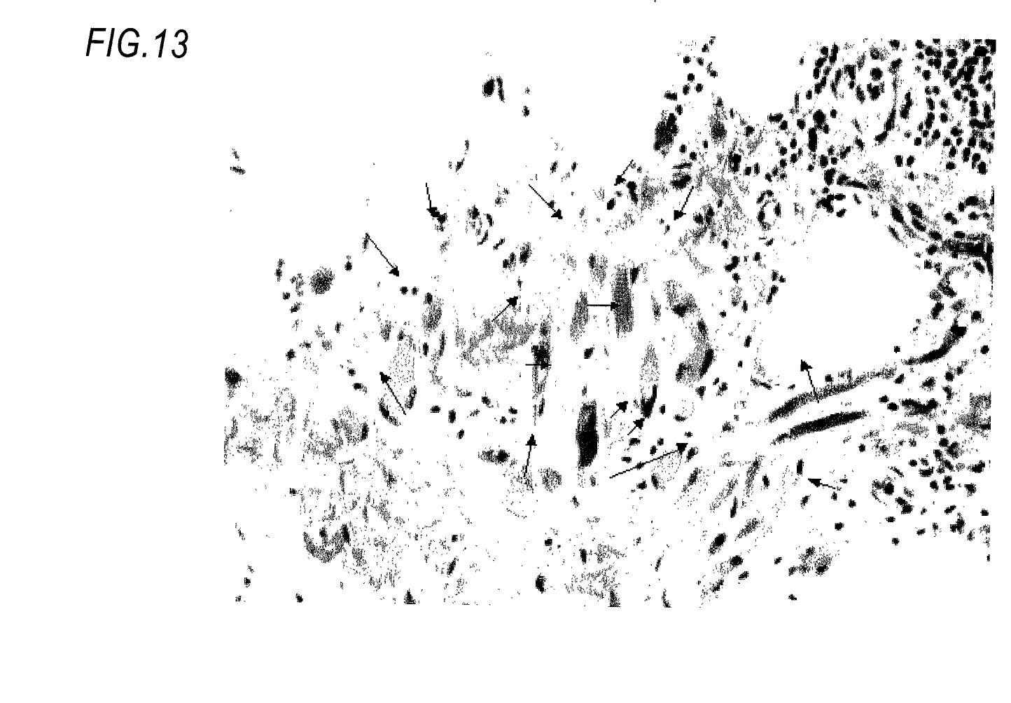

FIG. 12 is a micrograph showing a trichrome stain of surviving skeletal myofibers in patient heart. This area extends up from the epicardial surface of the myocardium into the epicardial fat. Blue stain represents collagen fibrils and red patches represent surviving myofibers. The boxed area is shown in FIG. 13 at higher magnification. Extensive evidence of myoblast engraftment was observed within adipose-rich regions as well as other regions of the heart. No differences in cell survival or phenotype were observed when results obtained from different injection sites were compared.

FIG. 13 is a micrograph showing a trichrome stain of surviving skeletal myofibers shown at 200×magnification. The blue staining area represents an area of collagen fibril deposition typical of scarred myocardium. The red stained areas marked by arrows show the myofibers, some of which show a striated appearance consistent with skeletal myofiber morphology.

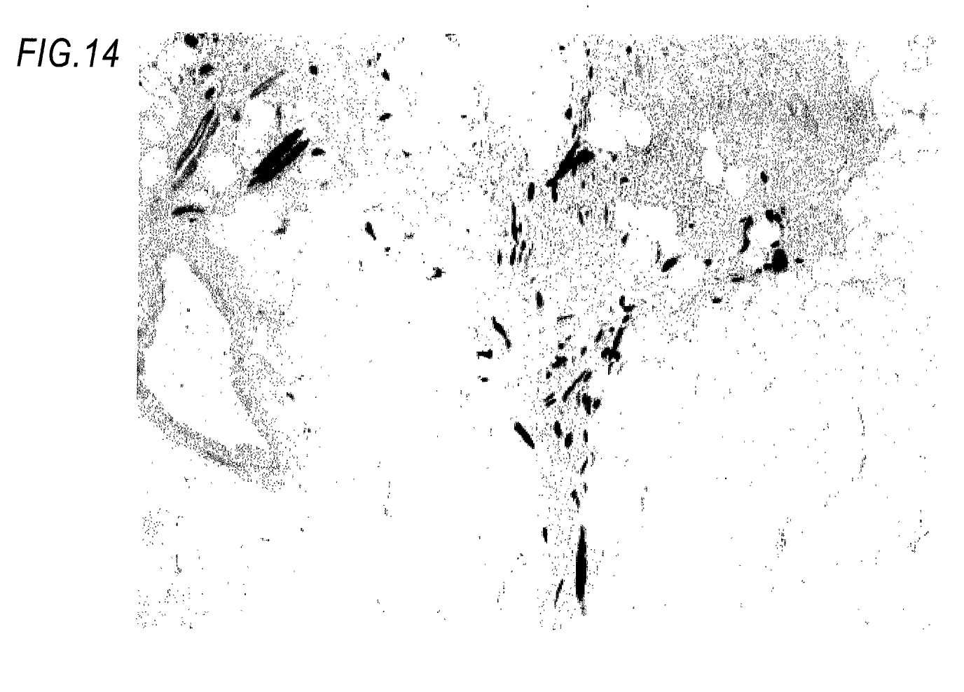

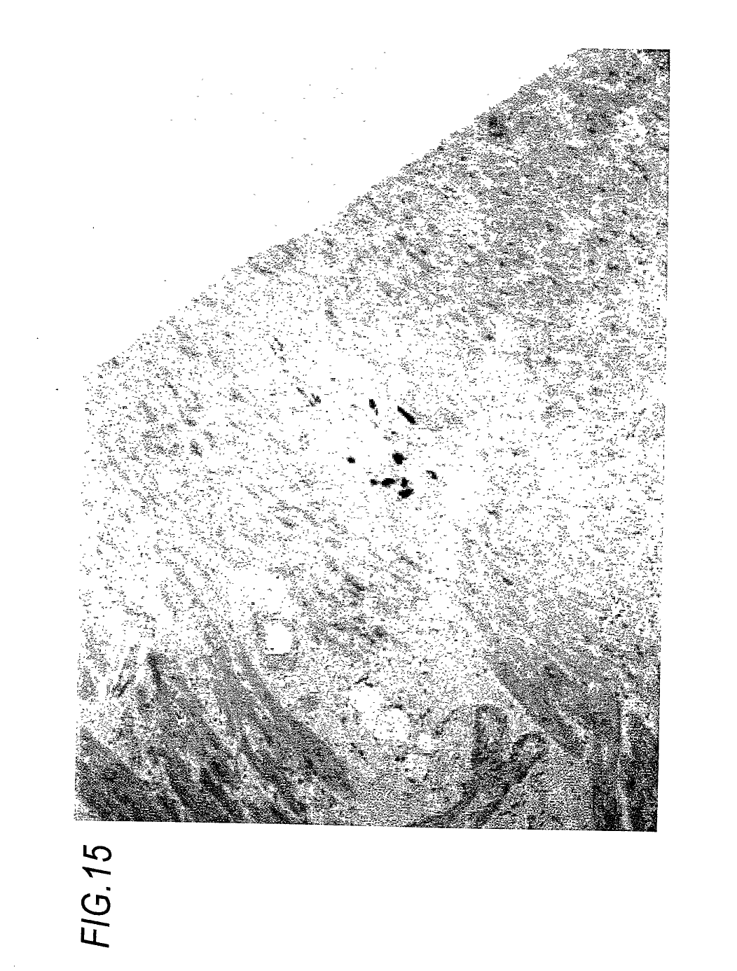

FIG. 14 shows staining of skeletal muscle fibers with skeletal muscle specific myosin indicating survival and differentiation of skeletal myoblasts within the heart. FIG. 15 shows muscle specific myosin staining of surviving skeletal muscle fibers in heart tissue that received transplanted cells. The myofibers are shown in the myocardium close to the epicardial surface.

### EXAMPLE 11

### Lack of Evidence of Survival of Inadequate Number of Autologous Myoblasts Transplanted into Infarcted Human Myocardium

Materials and Methods

Study Subject and Protocol: The patient (subject JW-01 in Table 6) was a 43 year-old male with a history of cardiac dysfunction. The patient was evaluated and approved for heart transplantation and underwent study recruitment and muscle biopsy. The muscle biopsy was taken from the right quadriceps muscle under sterile conditions using local anesthetics. The muscle specimen was immediately placed in transport medium and sent to the GMP isolation facility.

Subsequent to removal of the muscle sample, the patient was evaluated and underwent HeartMate® LVAD (Thoratec, Inc.) implantation as a bridge to heart transplantation. At the time of LVAD implantation, multiple injections of autologous skeletal myoblasts were made in a similar fashion to that described in Example 9. The LVAD implant procedure was completed in the usual fashion. The patient recovered uneventfully and was discharged to home with LVAD support. Approximately 3 months following LVAD implantation, the patient underwent LVAD explantation and heart transplantation. At the time of operation, the portion of the heart demarcated by the surgical clips was excised, and stored in formalin solution prior to histological analysis. The patient improved and was discharged to home in satisfactory condition. The patient is alive and well 10 months following transplantation.

Preparation of the Autologous Skeletal Myoblasts and Histological Analysis and Immunohistochemical Techniques. In general, these were performed similarly to described in Example 9. However, because the patient received a heart transplant shortly after the muscle specimen was obtained, the time available for expansion of the skeletal myoblasts was limited. Therefore, at the time of transplantation, only a total of approximately 2.2×10⁶ cells could be delivered. The percent myoblasts was approximately 75% and a total of only 3 injections were performed due to the small number of cells. Quantitative assessments were performed as described in Example 9.

Results

Skeletal myoblasts were expanded in culture as described above. Prior to transplantation, the population of approximately 2.2×10⁶ cells contained only single cells and no fused myoblasts. The cell preparation was composed of approximately 75% myoblasts based on skeletal muscle-specific anti-NCAM mAb staining and FACS analysis, with the remainder of the cells being composed of fibroblasts. The final myoblast preparation was characterized further by demonstrating the capacity to fuse and form multinucleated myotubes.

Approximately 2.2×10⁶ cells were transplanted using multiple injections. At the time of orthotopic heart transplantation, approximately three months after cells were implanted, the explanted heart was fixed and sectioned. The tissue was analyzed as described in Example 9. No evidence of skeletal myoblast survival or engraftment was observed. This result is most likely due to the very small number of cells delivered to the heart. The lack of evidence of skeletal myoblast survival or engraftment following delivery of a small number of cells serves as a useful negative control, confirming that the results obtained for the hearts described in Examples 9 and 10 are indeed due to skeletal myoblast survival and differentiation. This result additionally confirms the importance of delivering an adequate number of cells to the heart.

Equivalents

Those skilled in the art will recognize, or be able to ascertain using no more than routine experimentation, many equivalents of the specific embodiments of the invention described herein. Such equivalents are intended to be encompassed by the following claims.

References

Apstein, C. S., O. H. Bing, and H. J. Levine. 1976. Cardiac muscle function during and after hypoxia: effects of glucose concentration, mannitol and isoproteronol. J Mol Cell Cardiol. 8:627.

Baily, R. G., J. C. Lehman, S. S. Gubin, and T. I. Musch. 1993. Non-invasive assessment of ventricular damage in rats with myocardial infarction. Cardiovasc Res. 27:851.

Blau, Helen and Hughes, Simon. Publication Date: 1990. International Publication No. WO 90/15863. (International Application No. PCT/US90/03352).

Chen G. and Quinn L. S. 1992. Partial characterization of skeletal myoblast mitogens in mouse crushed muscle extract. J. Cell Physiol. 153(3):563-74

Connelly, C. M., S. Ngoy, F. J. Schoen, and C. S. Apstein. 1992. Biomechanical properties of reperfused transmural myocardial infarcts in rabbits during the first week after infarction. Implications for left ventricular rupture. Circ Res. 71:401.

Desai et al. 1997. Cardiovascular Indexes in the Mouse at Rest and with Exercise: New Tools to Study Models of Cardiac Disease. Am. J. Physiol. 272:H 1053-1061.

Desai et al. 1999. Phospholamban Deficiency Does Not Compromise Exercise Capacity. Am. J. Physiol. 276:H1 172-177.

Eberli, F. R., F. Sam, S. Ngoy, C. S. Apstein, and W. S. Colucci. 1998. Left-ventricular structural and functional remodeling in the mouse after myocardial infarction: assessment with the isovolumetrically-contracting Langendorff heart. J Mol Cell Cardiol. 30: 1443.

Fewell et al. 1997. A Treadmill Exercise Regimen for Identifying Cardiovascular phenotypes in Transgenic Mice. Am. J. Physiol. 273:H1 595-605.

Field, L. J. 1997. Non-Human Mammal Having a Graft and Methods of Delivering Protein to Myocardial Tissue. U.S. Pat. No. 5,602,301 (filed Nov. 16, 1994).

Field, L. J. et al. Publication Date: 1995. International Publication No. WO 95/14079. (International Application No. PCT/US94/13141).

Jain, M. et al. 2000. Angiotensin II Receptor Blockade Attenuates the Deleterious Effects of Exercise Training on Post-MI Ventricular Remodelling in Rats. Cardiovasc. Res. 46:66-72.

Koh, G. Y. et al. 1993 Differentiation and Long-Term Survival of C2C12 Myoblast Grafts in Heart. J. Clin. Invest. 92:1548-1554.

Law, P. K. and Goodwin, T. G. 1992. Compositions for and Methods of Treating Muscle Degeneration and Weakness. U.S. Pat. No. 5,130,141 (filed May 30, 1991).

Li, R. K. et al. 1998. Cell therapy to repair broken hearts. Can J Cardiol; 14(5):735-744.

Li, R. K. et al. 1996. Human pediatric and adult ventricular cardiomyocytes in culture: assessment of phenotypic changes with passaging. Cardiovascular Research. 32:362-373.

Li, R. K. et al. 1997. Natural History of Fetal Rat Cardiomyocytes Transplanted Into Adult Rat Myocardial Scar Tissue. Circulation. 96(9):II-179-II-187.

Mannion, J. D. et al. 1986. Histochemical and Fatigue Characteristics of Conditioned Canine Latissimus Dorsi Muscle. Circulation Research.58(1):298-304.

Morrow, N. G. et al. 1990. Increased Expression of Fibroblast Growth Factors in a Rabbit Skeletal Muscle Model of Exercise Conditioning. J. Clin. Invest. 85:1816-1820.

Murry, C. E. et al. 1996. Skeletal Myoblast Transplantation for Repair of Myocardial Necrosis. J. Clin. Invest. 98(11):2512-2523.

Pfeffer et al. 1979. Myocardial Infarct Size and Function in Rats. Circ. Res. 44:503-512.

Pfeffer, P. F., and E. Thorsby. 1982. HLA-restricted cytotoxicity against male-specific (HY) antigen after acute rejection of an HLA-identical sibling kidney: clonal distribution of the cytotoxic cells. Transplantation. 33:52.

Robinson, S. W. et al. 1996. Arterial Delivery of Genetically Labelled Skeletal Myoblasts to the Murine Heart: Long-Term Survival and Phenotype Modification of Implanted Myoblasts. Cell Transplantation. 5(1):77-91.

Schweitzer, J. S. et al. 1987. Fibroblasts Modulate Expression of Thy-1 on the Surface of Skeletal Myoblasts. Experimental Cell Research. 172:1-20.

Taylor, D. A. et al. 1997. Delivery of Primary Autologous Skeletal Myoblasts into Rabbit Heart by Coronary Infusion: A Potential Approach to Myocardial Repair. Proceedings of the Association of American Physicians. 109(3):245-253.

Taylor, D. A. et al. 1998. Regenerating functional myocardium: Improved performance after skeletal myoblast transplantation. Nature Medicine. 4(8):929-933.

Trueblood et al. 1999. Relationship Between Exercise Intolerance and Myocardial Remodeling Post-MI in the Rat. Circulation. 100:I-55. Abstract.

Zelenika, D., E. Adams, A. Mellor, E. Simpson, P. Chandler, B. Stockinger, H. Waldmann, and S. P. Cobbold. 1998. Rejection of H-Y disparate skin grafts by monospecific CD4+ Th1 and Th2 cells: no requirement for CD8+ T cells or B cells. J Immunol. 161: 1868.

1. A method of treating a dysfunctional heart comprising:
identifying a subject in need of treatment for cardiac dysfunction; and
delivering a composition comprising skeletal myoblasts to the subject's heart, wherein at least a portion of the skeletal myoblasts, or cells to which the skeletal myoblasts give rise, survive in the heart after delivery and express therein a marker characteristic of skeletal myoblast survival or differentiation.

2. The method of claim 1, wherein the composition further comprises fibroblasts.

3. The method of claim 1, wherein the marker is characteristic of skeletal myoblasts, skeletal myotubes, skeletal myofibers, or of skeletal myotube fusion.

4. The method of claim 3, wherein the marker is skeletal muscle-specific myosin heavy chain.

5. The method of claim 1, wherein the marker is desmin.

6. The method of claim 1, wherein the marker distinguishes skeletal myoblasts or cells derived from skeletal myoblasts from cardiac cells.

7. The method of claim 1, wherein the marker distinguishes skeletal myoblasts from myotubes or myofibers.

8. The method of claim 7, wherein the marker is selected from the group consisting of myoD, myogenin, myf-5, and NCAM.

9. The method of claim 1, wherein the subject is a human.

10. The method of claim 1, wherein the subject suffers from ischemic heart disease.

11. The method of claim 1, wherein the subject's heart has suffered damage caused by a viral infection.

12. The method of claim 1, wherein the subject's heart has suffered damage caused by an exogenous compound.

13. The method of claim 1, wherein the subject's heart has suffered damage mediated by an immune system activity.

14. The method of claim 1, wherein the subject suffers from congestive heart failure.

15. The method of claim 1, wherein the subject's heart has suffered damage at least 1 hour prior to delivery of the composition.

16. The method of claim 1, wherein the subject's heart has suffered damage at least 24 hours prior to delivery of the composition.

17. The method of claim 1, wherein the subject's heart has suffered damage at least 1 month prior to delivery of the composition.

18. The method of any of claims 15, 16, or 17, wherein the damage is ischemic damage.

19. The method of claim 1, wherein the subject's heart has suffered damage at least 6 months prior to delivery of the composition.

20. The method of claim 1, wherein the subject's heart has suffered damage at least 1 year prior to delivery of the composition.

21. The method of claim 19 or claim 20, wherein the damage is ischemic damage.

22. The method of claim 1, wherein the composition is delivered to myocardial scar tissue.

23. The method of claim 1, wherein the composition is delivered to myocardial scar tissue and to adjacent myocardial tissue not showing evidence of scarring.

24. The method of claim 1, wherein the composition is delivered to an adipose-rich region of the heart.

25. The method of claim 1, wherein at least 1×10⁶ skeletal myoblasts are delivered.

26. The method of claim 1, wherein between approximately 10⁶ and 10⁷ skeletal myoblasts are delivered.

27. The method of claim 1, wherein between approximately 10⁷ and 10⁸ skeletal myoblasts are delivered.

28. The method of claim 1, wherein between approximately 10⁸ and 10⁹ skeletal myoblasts are delivered.

29. The method of claim 1, wherein between approximately 10⁹ and 10¹⁰ skeletal myoblasts are delivered.

30. The method of claim 1, wherein approximately 300×10⁶ skeletal myoblasts are delivered.

31. The method of claim 1, wherein the skeletal myoblasts are delivered at a concentration of approximately 8×10⁷ cells/ml.

32. The method of claim 1, wherein the skeletal myoblasts are delivered at a concentration of up to 16×10⁷ cells/ml.

33. The method of claim 1, wherein the composition further comprises fibroblasts, and wherein at least 1×10^{6,} between approximately 10⁶ and 10⁷, between approximately 10⁷ and 10⁸, between approximately 10⁸ and 10⁹, or between approximately 109 and 100 cells are delivered.

34. The method of claim 1, wherein the composition further comprises fibroblasts, and wherein the skeletal myoblasts and fibroblasts are delivered at a total concentration of approximately 8×10⁷ cells/ml.

35. The method of claim 1, wherein the composition further comprises fibroblasts, and wherein the skeletal myoblasts and fibroblasts are delivered at a total concentration of up to 16×10⁷ cells/ml.

36. The method of claim 1, wherein the composition is delivered to the endocardium or epicardium.

37. The method of claim 1, wherein the composition is delivered intraarterially.

38. The method of claim 1, wherein the composition is delivered intravenously.

39. The method of claim 1, wherein the composition is delivered to the heart via a catheter that is inserted into the venous system.

40. The method of claim 1, wherein the composition comprises at least 30% skeletal myoblasts.

41. The method of claim 1, wherein the composition comprises between approximately 30% and 50% skeletal myoblasts.

42. The method of claim 1, wherein the composition comprises between approximately 50% and 60% skeletal myoblasts.

43. The method of claim 1, wherein the composition comprises between approximately 60% and 75% skeletal myoblasts.

44. The method of claim 1, wherein the composition comprises between approximately 75% and 90% skeletal myoblasts.

45. The method of claim 1, wherein the composition comprises between approximately 90% and 95% skeletal myoblasts.

46. The method of claim 1, wherein the composition comprises between approximately 95% and 99% skeletal myoblasts.

47. The composition of claim 1, wherein the composition comprises at least 99% skeletal myoblasts.

48. The method of claim 1, wherein the composition further comprises fibroblasts.

49. The method of claim 48, wherein the composition comprises at least 5% fibroblasts, at least 10% fibroblasts, at least 25% fibroblasts, at least 50% fibroblasts, or at least 70% fibroblasts.

50. The method of claim 1, wherein the composition comprises less than approximately 1% myotubes.

51. The method of claim 1, wherein the composition comprises less than approximately 0.5% myotubes.

52. The method of claim 1, wherein the composition is essentially free of myotubes.

53. The method of claim 1, wherein the composition comprises less than approximately 1% endothelial cells.

54. The method of claim 1, wherein the composition comprises less than approximately 0.5% endothelial cells.

55. The method of claim 1, wherein the composition is essentially free of endothelial cells.

56. The method of claim 1, wherein the skeletal myoblasts are autologous.

57. The method of claim 1, wherein the skeletal myoblasts, or cells to which the myoblasts give rise, survive for at least 30 days

58. The method of claim 1, wherein the skeletal myoblasts, or cells to which the myoblasts give rise, survive for at least 60 days.

59. The method of claim 1, wherein the skeletal myoblasts, or cells to which the skeletal myoblasts give rise, survive for at least 90 days.

60. The method of claim 1, wherein the skeletal myoblasts, or cells to which the skeletal myoblasts give rise, survive for at least 1 year.

61. The method of claim 1, wherein small vessel formation occurs at or in the vicinity of the surviving skeletal myoblasts or cells to which the skeletal myoblasts give rise.

62. The method of claim 61, wherein small vessel formation is evidenced by expression of an endothelial cell marker.

63. The method of claim 1, wherein the composition is delivered in conjunction with a procedure in which the subject receives a left ventricular assist device.

64. The method of claim 1, wherein the composition is delivered in conjunction with a procedure in which the subject receives a coronary artery bypass graft.

65. The method of claim 1, wherein the composition is delivered in conjunction with a procedure in which the subject receives a valve replacement.

66. A method of preparing a composition for transplantation into a subject's heart comprising steps of:
obtaining a sample of muscle tissue from a subject;
isolating a population of cells from the sample, wherein the population of cells comprises skeletal myoblasts;
expanding the population in culture; and
preparing the population that results from the expanding step to produce a transplantable composition comprising skeletal myoblasts characterized by the ability to survive, or to give rise to cells that survive, in the subject's heart after delivery and express therein a marker characteristic of skeletal myoblast survival or differentiation.

67. The method of claim 66, wherein the population prepared in the preparing step further comprises fibroblasts.

68. The method of claim 66, wherein the cells are maintained in a subconfluent state during the expanding step.

69. The method of claim 68, wherein the cells are maintained at less than approximately 75% confluence during the expanding step.

70. The method of claim 66, wherein the isolating step includes digesting the sample in a digestion mixture comprising at least two proteases.

71. The method of claim 70, wherein the digestion mixture comprises EDTA.

72. The method of claim 70, wherein the proteases are selected from the group consisting of carboxypeptidase, caspase, chymotrypsin, collagenase, elastase, endoproteinase, leucine aminopeptidase, papain, pronase, and trypsin.

73. The method of claim 66, wherein the expanding step comprises maintaining the population of cells in culture for less than approximately 50 doubling times.

74. The method of claim 66, wherein the expanding step comprises maintaining the population of cells in culture for between approximately 5 and 15 doubling times.

75. The method of claim 66, wherein the preparing step comprises combining a population of cells comprising skeletal myoblasts with a population of cells comprising fibroblasts.

76. The method of claim 75, wherein the population of cells comprising skeletal fibroblasts is obtained by expanding, in culture, a population of cells isolated from the sample.

77. The method of claim 66 or claim 75, wherein the preparing step comprises sorting the cells.

78. The method of claim 77, wherein one or both of the isolating step or the preparing step comprises performing flow cytometry or fluorescence activated cell sorting.

79. The method of claim 66, wherein the subject is a human.

80. A transplantable composition comprising skeletal myoblasts, wherein the composition is characterized by an ability, when delivered to a subject's heart, to survive in the heart after delivery and there express a marker characteristic of skeletal myoblast survival or differentiation.

81. The transplantable composition of claim 80, wherein the composition further comprises fibroblasts.

82. The transplantable composition of claim 80, wherein the marker is characteristic of skeletal myoblasts, skeletal myotubes, skeletal myotube fusion, or skeletal myofibers.

83. A transplantable composition prepared according to the method of claim 66.
